SEQUENCE LISTING (45 sequences)

SEQ ID NO: 1 - DNA, 51 bp, synthetic construct
  source 1..51: mol_type = other DNA; note = Primer HemH fw
gtttaactttaagaaggagatatacatatgcgtcagactaaaaccggtatc

SEQ ID NO: 2 - DNA, 51 bp, synthetic construct
  source 1..51: mol_type = other DNA; note = Primer HemH rv
ccggatctcaatggtgatggtgatggtggcgatacgcggcaacaagattag

SEQ ID NO: 3 - DNA, 34 bp, synthetic construct
  source 1..34: mol_type = other DNA; note = Primer L13X fw
gtatcctgctggcaaacndtggtacgcccgatgc

SEQ ID NO: 4 - DNA, 34 bp, synthetic construct
  source 1..34: mol_type = other DNA; note = Primer L13X fw
gtatcctgctggcaaacvhgggtacgcccgatgc

SEQ ID NO: 5 - DNA, 34 bp, synthetic construct
  source 1..34: mol_type = other DNA; note = Primer L13X fw
gtatcctgctggcaaacttgggtacgcccgatgc

SEQ ID NO: 6 - DNA, 32 bp, synthetic construct
  source 1..32: mol_type = other DNA; note = Primer L13X rv
gtttgccagcaggataccggttttagtctgac

SEQ ID NO: 7 - DNA, 40 bp, synthetic construct
  source 1..40: mol_type = other DNA; note = Primer F31X fw
ggtaaaacgctatctgaaacaandtttaagcgacagacgc

SEQ ID NO: 8 - DNA, 40 bp, synthetic construct
  source 1..40: mol_type = other DNA; note = Primer F31X fw
ggtaaaacgctatctgaaacaavhgttaagcgacagacgc

SEQ ID NO: 9 - DNA, 40 bp, synthetic construct
  source 1..40: mol_type = other DNA; note = Primer F31X fw
ggtaaaacgctatctgaaacaattgttaagcgacagacgc

SEQ ID NO: 10 - DNA, 34 bp, synthetic construct
  source 1..34: mol_type = other DNA; note = Primer F31X rv
ttgtttcagatagcgttttaccgcttcaggtgtg

SEQ ID NO: 11 - DNA, 33 bp, synthetic construct
  source 1..33: mol_type = other DNA; note = Primer R57X fw
cgtgattttgccgctgndttcgccgcgtgtggc

SEQ ID NO: 12 - DNA, 33 bp, synthetic construct
  source 1..33: mol_type = other DNA; note = Primer R57X fw
cgtgattttgccgctgvhgtcgccgcgtgtggc

SEQ ID NO: 13 - DNA, 32 bp, synthetic construct
  source 1..32: mol_type = other DNA; note = Primer R57X fw
cgtgattttgccgctgttgcgccgcgtgtggc

SEQ ID NO: 14 - DNA, 30 bp, synthetic construct
  source 1..30: mol_type = other DNA; note = Primer R57X rv
gcggcaaaatcacgccgcgcagcaatggcc

SEQ ID NO: 15 - DNA, 30 bp, synthetic construct
  source 1..30: mol_type = other DNA; note = Primer Y128X fw
gtggtgctgccgcttndtccgcaatactcc

SEQ ID NO: 16 - DNA, 30 bp, synthetic construct
  source 1..30: mol_type = other DNA; note = Primer Y128X fw
gtggtgctgccgcttvhgccgcaatactcc

SEQ ID NO: 17 - DNA, 30 bp, synthetic construct
  source 1..30: mol_type = other DNA; note = Primer Y128X fw
gtggtgctgccgcttttgccgcaatactcc

SEQ ID NO: 18 - DNA, 29 bp, synthetic construct
  source 1..29: mol_type = other DNA; note = Primer Y128X rv
gcggcagcaccacaatatgatctacatgc

SEQ ID NO: 19 - DNA, 44 bp, synthetic construct
  source 1..44: mol_type = other DNA; note = Primer S134X fw
gtgctgccgctttatccgcaatactcctgtndtacggtcggtgc

SEQ ID NO: 20 - DNA, 44 bp, synthetic construct
  source 1..44: mol_type = other DNA; note = Primer S134X fw
gtgctgccgctttatccgcaatactcctgtvhgacggtcggtgc

SEQ ID NO: 21 - DNA, 44 bp, synthetic construct
  source 1..44: mol_type = other DNA; note = Primer S134X fw
gtgctgccgctttatccgcaatactcctgtttgacggtcggtgc

SEQ ID NO: 22 - DNA, 44 bp, synthetic construct
  source 1..44: mol_type = other DNA; note = Primer S134X rv
ggataaagcggcagcaccacaatatgatctacatgctctgccag

SEQ ID NO: 23 - DNA, 35 bp, synthetic construct
  source 1..35: mol_type = other DNA; note = Primer H194X fw
ctgctgctgctctctndttggggcattccccagcg

SEQ ID NO: 24 - DNA, 35 bp, synthetic construct
  source 1..35: mol_type = other DNA; note = Primer H194X fw
ctgctgctgctctctvhgtggggcattccccagcg

SEQ ID NO: 25 - DNA, 35 bp, synthetic construct
  source 1..35: mol_type = other DNA; note = Primer H194X fw
ctgctgctgctctctttgtggggcattccccagcg

SEQ ID NO: 26 - DNA, 27 bp, synthetic construct
  source 1..27: mol_type = other DNA; note = Primer H194X rv
taagagagcagcagcagatccggttcg

SEQ ID NO: 27 - DNA, 37 bp, synthetic construct
  source 1..37: mol_type = other DNA; note = Primer W242X fw
cgctttggtcgggaacccndtctgatgccttataccg

SEQ ID NO: 28 - DNA, 37 bp, synthetic construct
  source 1..37: mol_type = other DNA; note = Primer W242X fw
cgctttggtcgggaacccvhgctgatgccttataccg

SEQ ID NO: 29 - DNA, 37 bp, synthetic construct
  source 1..37: mol_type = other DNA; note = Primer W242X fw
cgctttggtcgggaacccttgctgatgccttataccg

SEQ ID NO: 30 - DNA, 38 bp, synthetic construct
  source 1..38: mol_type = other DNA; note = Primer W242X rv
ggttcccgaccaaagcgcgactgaaaggtcatcatcac

SEQ ID NO: 31 - DNA, 30 bp, synthetic construct
  source 1..30: mol_type = other DNA; note = Primer F269X fw
ggtgatgtgcccgggcndtgctgcggattg

SEQ ID NO: 32 - DNA, 30 bp, synthetic construct
  source 1..30: mol_type = other DNA; note = Primer F269X fw
ggtgatgtgcccgggcvhggctgcggattg

SEQ ID NO: 33 - DNA, 30 bp, synthetic construct
  source 1..30: mol_type = other DNA; note = Primer F269X fw
ggtgatgtgcccgggcttggctgcggattg

SEQ ID NO: 34 - DNA, 26 bp, synthetic construct
  source 1..26: mol_type = other DNA; note = Primer F269X rv
gcccgggcacatcacctgtatatgac

SEQ ID NO: 35 - DNA, 29 bp, synthetic construct
  source 1..29: mol_type = other DNA; note = Primer C273X fw
gctttgctgcggatndtctggagacgctg

SEQ ID NO: 36 - DNA, 29 bp, synthetic construct
  source 1..29: mol_type = other DNA; note = Primer C273X fw
gctttgctgcggatvhgctggagacgctg

SEQ ID NO: 37 - DNA, 29 bp, synthetic construct
  source 1..29: mol_type = other DNA; note = Primer C273X fw
gctttgctgcggatttgctggagacgctg

SEQ ID NO: 38 - DNA, 22 bp, synthetic construct
  source 1..22: mol_type = other DNA; note = Primer C273X rv
atccgcagcaaagcccgggcac

SEQ ID NO: 39 - DNA, 35 bp, synthetic construct
  source 1..35: mol_type = other DNA; note = Primer E275X fw
gctgcggattgtctgndtacgctggaagagattgc

SEQ ID NO: 40 - DNA, 35 bp, synthetic construct
  source 1..35: mol_type = other DNA; note = Primer E275X fw
gctgcggattgtctgvhgacgctggaagagattgc

SEQ ID NO: 41 - DNA, 35 bp, synthetic construct
  source 1..35: mol_type = other DNA; note = Primer E275X fw
gctgcggattgtctgttgacgctggaagagattgc

SEQ ID NO: 42 - DNA, 24 bp, synthetic construct
  source 1..24: mol_type = other DNA; note = Primer E275X rv
cagacaatccgcagcaaagcccgg

SEQ ID NO: 43 - DNA, 981 bp, Escherichia coli
  source 1..981: mol_type = genomic DNA; note = EcHemH gene
atgcgtcagactaaaaccggtatcctgctggcaaacctgggtacgcccgatgcccccacacctgaagcggtaaaacgctatctgaaacaatttttaagcgacagacgcgtggttgatacctcacggttgttatggtggccattgctgcgcggcgtgattttgccgctgcgctcgccgcgtgtggcgaagctgtatgcctctgtctggatggaaggtggctcgccgctgatggtttacagccgtcagcaacagcaggcgctggcacaacgtttaccggagacgcccgtagcgctgggaatgagctacggctcgccatcactggaaagcgccgtagatgaactcctggcagagcatgtagatcatattgtggtgctgccgctttatccgcaatactcctgttcaacggtcggtgcggtatgggatgaactggcacgcattctggcgcgcaaacgtagcattccggggatatcgtttattcgtgattacgctgataaccacgattacattaatgcactggcgaacagcgtacgcgcttcttttgccaaacatggcgaaccggatctgctgctgctctcttatcatggcattccccagcgttatgcagatgaaggcgatgattacccgcaacgttgccgcacaacgactcgcgaactggcttccgcactggggatggcaccggaaaaagtgatgatgacctttcagtcgcgctttggtcgggaaccctggctgatgccttataccgacgaaacgctgaaaatgctcggagaaaaaggcgtaggtcatatacaggtgatgtgcccgggctttgctgcggattgtctggagacgctggaagagattgccgagcaaaaccgtgaggtcttcctcggtgccggcgggaaaaaatatgaatatattccagcgcttaatgccacgccggaacatattgaaatgatggctaatcttgttgccgcgtatcgccaccatcaccatcaccattga

SEQ ID NO: 44 - DNA, 924 bp, Enterobacter lignolyticus
  source 1..924: mol_type = genomic DNA; note = DyP gene
atgtcacaagtgcagtctggcattcttccagagcactgtcgtgcagccatctggatcgaggcaaacgtgaaaggggatgtcaatgcactgcgcgagtgctcaaaagtctttgcagataaattagctggtttcgaggcacagtttccagacgcacatcttggagcggtcgttgcctttggacatgacacatggcgtgctttgtcggggggggtaggggccgaggaattgaaggatttcacgccttatggcaagggtttggctccagccactcagtacgatgtccttatccatattttatctctgcgtcacgacgtaaatttttccgtcgcccaagcggccatggccgcttttggggatgctgtggaggtgaaagaagagattcacggctttcgctgggtcgaagaacgtgacttatctgggttcgtagatgggacagaaaatccagcgggggaagaaacacgccgtgaagtcgccgttattaaagatggggttgacgccggaggcagttacgtttttgtgcaacgctgggagcataacttaaaacaacttaaccgtatgtcagttcacgatcaggaaatgatgatcggtcgtacaaaagttgcaaacgaagaaattgatggcgatgagcgcccagagacaagccacttaacgcgtgtcgaccttaaagagaacggaaaaggcttaaaaattgttcgtcagagcttgccgtacggtactgccagcggcacgcatggattgtatttctgcgcgtattgcgcgcgcttgtataatatcgagcagcagttgctgtccatgttcggtgacacggacggaaaacgtgatgcaatgcttcgcttcaccaagcctgttacagggggttactactttgcgccgtcgttagataaattacttgccttgctcgagcaccatcaccatcaccattga

SEQ ID NO: 45 - DNA, 1131 bp, Sulfolobus acidocaldarius
  source 1..1131: mol_type = genomic DNA; note = CYP119 gene
atgtacgattggttttcggagatgcgtaaaaaagatcctgtttactacgatggtaatatttggcaagtattttcatatcgctacaccaaggaggtattaaacaatttttcaaaattttcttccgatttaacagggtaccacgagcgccttgaggacttgcgcaatggaaagatccgctttgacatcccaacccgctataccatgcttacgtcggatccgccgctgcatgacgaattgcgtagtatgagtgccgatatcttttctccgcaaaagttgcaaaccctggaaacttttattcgcgaaacgacccgtagtttattggactcgattgaccctcgcgaggacgatattgttaagaagctggccgtgccccttccaattatcgtgatttccaaaatcctgggtctgccaatcgaggacaaagagaagttcaaggaatggagcgaccttgtagcgtttcgtctgggcaaacctggcgagatcttcgaattaggaaagaagtatctggaattgatcggatatgtgaaagaccatcttaattcaggtacggaagtggtcagccgcgtagttaattcgaatctttctgacattgaaaaattaggatatatcattttattattgattgccgggaacgagacgactactaatctgattagcaattcagttattgacttcacgcgcttcaacttgtggcaacgcattcgcgaagaaaacctttacctgaaagctattgaagaggctcttcgctattcacccccggttatgcgcacagttcgtaaaaccaaggagcgtgtaaagttgggcgaccagacgattgaggaaggagaatacgtgcgcgtctggattgcatcagcgaaccgcgacgaagaggtctttcacgacggagaaaagttcattccagatcgtaacccgaatcctcatctgagtttcgggagtgggatccacctgtgcctgggggcacctttggcacgtttggaagcccgtattgccatcgaggagttctcgaagcgttttcgtcacatcgaaattcttgacacagaaaaggtgccaaacgaagtgttgaacgggtataagcgtttggtagttcgtctgaagagcaatgagctcgagcaccatcaccatcaccattga

(19) United States
(12) Patent Application Publication
(10) Pub. No.: US 2026/0022410 A1
(43) Pub. Date: Jan. 22, 2026

(54) Method To Make Co-, and Ni-Substituted Heme Proteins

(71) Applicant: Wisconsin Alumni Research Foundation, Madison, WI (US)

(72) Inventors: Andrew Buller, Madison, WI (US); Lydia Perkins, Madison, WI (US); Brian Weaver, Madison, WI (US); Judith Burstyn, Madison, WI (US)

(73) Assignee: Wisconsin Alumni Research Foundation, Madison, WI (US)

(21) Appl. No.: 18/779,932

(22) Filed: Jul. 22, 2024

(51) Int. Cl. C12P 17/16; C12N 9/88; C12P 21/00

(52) U.S. Cl. CPC C12P 17/165; C12N 9/88; C12P 21/00; C12Y 499/01001

(57) ABSTRACT

A method to make Co- or Ni-substituted protoporphyrin IX by incubating an E. coli strain in a rich medium containing iron ions and added Co²⁺ or Ni²⁺ ions, for a time and under conditions wherein the E. coli strain produces Co- or Ni-substituted protoporphyrin IX. The E. coli host may be transformed to contain and express a gene construct encoding a mutant EcHemH including a L13R mutation.

### FEDERAL FUNDING STATEMENT

This invention was made with government support under 2237213 awarded by the National Science Foundation. The government has certain rights in the invention.

### SEQUENCE LISTING

The instant application contains a Sequence Listing which has been submitted in an XML file with the USPTO through Patent Center and is hereby incorporated by reference in its entirety. The Sequence Listing XML, created on Oct. 10, 2024, is named “SEQ_LIST--09824515-P240072US01.xml” and is 51,959 bytes in size.

### BACKGROUND

Proteins bearing unnatural metallocofactors are useful tools to study metalloprotein function, expand the reactivity of natural proteins, and catalyze non-natural, biological reactions. In particular, metal-substituted hemoproteins have been explored extensively because they have unique reactivity, can function as spectroscopic probes, and have unique imaging properties. See, for example, Bruha et al. “Resonance Raman Studies of Dioxygen Adducts of Co-Substituted Heme Proteins and Model Compounds. Vibrationally Coupled Dioxygen and the Issue of Multiple Structures and Distal Side Hydrogen Bonding,” J. Am. Chem. Soc. 1988, 110 (18), 6006-60014. While there are diverse methods to generate proteins loaded with unnatural metalloporphyrin cofactors both in vivo and in vitro, many of these strategies require exogenous synthesis of the unnatural metalloporphyrin and rely on low-efficiency methods for inserting the cofactor into the protein. See, for example, Liu et al. “Assembly and Evolution of Artificial Metalloenzymes within E. coli Nissle 1917 for Enantioselective and Site-Selective Functionalization of C—H and C═C Bonds,” J. Am. Chem. Soc. 2022, 144 (2), 883-890. Perkins et al. (2021) reported a method for the de novo biosynthesis of the non-native heme analog, cobalt protoporphyrin IX (CoPPIX), in E. coli BL21 (DE3) and its incorporation into hemoproteins. See Perkins et al. “De Novo Biosynthesis of a Nonnatural Cobalt Porphyrin Cofactor in E. coli and Incorporation into Hemoproteins,” Proc. Natl. Acad. Sci. 2021, 118 (16), 2021. The fully biosynthetic method described by Perkins et al. streamlined producing CoPPIX-bearing artificial metalloproteins.

Nature typically assembles metalloproteins and metallocofactors with impressive fidelity. Nevertheless, there are reports of improperly metalated heme cofactors occurring spontaneously in living systems. In humans, zinc protoporphyrin IX (ZnPPIX) is a biomarker for altered iron homeostasis and is implicated in some thalassemias. Labbé, et al. “Zinc Protoporphyrin: A Metabolite with a Mission,” Clin. Chem. 1999, 45 (12), 2060-2072.

Majtan et al. found that E. coli BL21-Rosetta2(DE3), when passaged for several days through iron-poor, cobalt-rich media, adventitiously biosynthesized and incorporated CoPPIX into both endogenous and heterologously expressed hemoproteins. Majtan, et al. “Effect of Cobalt on Escherichia coli Metabolism and Metalloporphyrin Formation,” BioMetals 2011, 24 (2), 335-347. Majtan, et al. “Purification and Characterization of Cystathionine β-Synthase Bearing a Cobalt Protoporphyrin,” Arch. Biochem. Biophys. 2011, 508 (1), 25-30.

Perkins et al. (supra) reported an expansion of this finding by showing that the standard laboratory strain E. coli BL21(DE3) innately possesses the ability to biosynthesize and incorporate CoPPIX, without passaging. Addition of an inexpensive cobalt salt to iron-deficient minimal media was sufficient to produce cobalt-substituted hemoproteins from diverse fold families with >95% cobalt loadings. Others similarly found that ZnPPIX can be over-produced by an engineered B-derived strain of E. coli under iron-poor, zinc-rich fermentation conditions. Choi, et al. “Production of Zinc Protoporphyrin IX by Metabolically Engineered Escherichia coli,” Biotechnol. Bioeng. 2022, 119 (11), 3319-3325. These reports indicate that heme biosynthesis in E. coli may be manipulated toward production of non-natural metalloporphyrin cofactors.

However, these cases of alternative heme metalation in E. coli require iron-deficient growth conditions to reduce production of the native heme cofactor. Such conditions severely limit cell growth and, correspondingly, the titers of expressed cobalt-substituted hemoproteins are also very low when compared to standard expression of hemoproteins. (See Perkins et al., supra) Thus, there remains a long-felt and unmet need for a more efficient process of synthesizing the non-native cofactors CoPPIX, NiPPIX, CuPPIX, and ZnPPIX and incorporating the non-native cofactors into heme-containing proteins.

### SUMMARY OF THE INVENTION

Disclosed herein is a method to make Co- or Ni-substituted protoporphyrin IX. The method comprises incubating an E. coli strain in a rich medium containing added Co²⁺ or Ni²⁺ ions, for a time and under conditions wherein the E. coli strain produces Co- or Ni-substituted protoporphyrin IX. Preferably, the E. coli strain is selected from the group consisting of E. coli BL21(DE3) and E. coli JM109(DE3). The rich medium may (or may not) contain iron ions.

In one version of the method, the E. coli strain does not overexpress E. coli ferrochelatase (“EcHemH”). In a different version, the E. coli strain overexpresses EcHemH. In yet another version of the method, the E. coli strain is transformed to contain and express an exogenous gene encoding EcHemH with a L13R mutation.

As noted above, the rich medium may contain iron. The rich medium may also comprise δ-aminolevulinic acid.

The rich medium preferably comprises from about 0.1 to about 3 mM Co²⁺ or Ni²⁺ ions, more preferably from about 0.5 to about 2 mM Co²⁺ or Ni²⁺ ions, more preferably still about 1 mM Co²⁺ or Ni²⁺ ions.

Also disclosed herein is a method of incorporating the Co- or Ni-substituted protoporphyrin IX into a polypeptide or protein.

Further disclosed herein is an E. coli host transformed to contain and express a gene construct encoding a mutant EcHemH including a L13R mutation. The E. coli is preferably selected from the group consisting of E. coli BL21(DE3) and E. coli JM109(DE3).

### ABBREVIATIONS AND DEFINITIONS

δALA=δ-aminolevulinic acid:

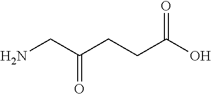

DyP=dye-decolorizing peroxidase. EcHemH=E. coli ferrochelatase. EPR=electron paramagnetic resonance. ICP-MS=inductively coupled plasma-mass spectrometry. IPTG=Isopropyl β-d-1-thiogalactopyranoside. LB=Luria-Bertani [broth]. MOPS=3-(N-morpholino)propanesulfonic acid. OD=optical density. PPIX=protoporphyrin IX. SIR=susceptible-infectives-recovered. SUMS=substrate multiplexed screening. TB=Terrific Broth.
Tris=tris(hydroxymethyl)aminomethane
UPLC-MS=ultra-performance liquid chromatography-mass spectrometry

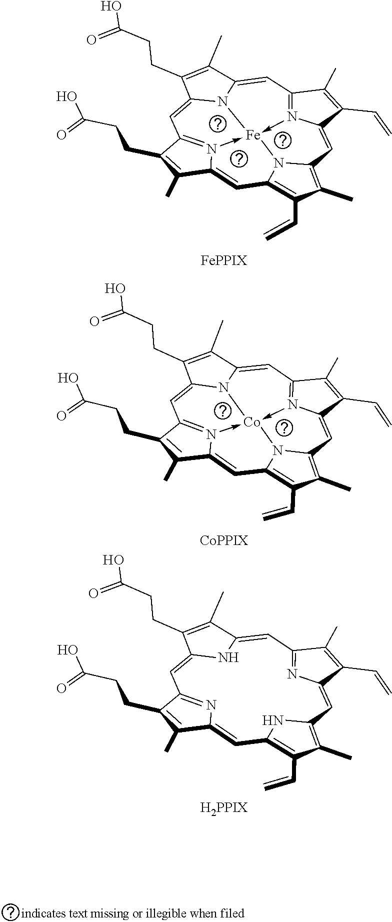

NiPPIX, CuPPIX, and ZnPPIX are analogous to CoPPIX, but include as the complexed metal atom nickel, copper, and zinc, respectively.

All references to singular characteristics or limitations of the disclosed method shall include the corresponding plural characteristic or limitation, and vice-versa, unless otherwise specified or clearly implied to the contrary by the context in which the reference is made. The indefinite articles “a” and “an” mean “one or more.” The word “or” is defined inclusively and means “and/or” unless explicitly stated otherwise.

All combinations of method steps disclosed herein can be performed in any order, unless otherwise specified or clearly implied to the contrary by the context in which the referenced combination is made.

The method disclosed herein can comprise, consist of, or consist essentially of the essential elements and steps described herein, as well as any additional or optional ingredients, components, or limitations described herein or otherwise useful in producing metallo-proteins in E. coli hosts.

### BRIEF DESCRIPTION OF DRAWINGS

FIG. 1 is a schematic diagram of EcHemH reacting with ferrous iron and protoporphyrin IX (“PPIX”) to make heme b.

FIG. 2A shows data concerning E. coli ferrochelatase (EcHemH)-catalyzed insertion of native Fe²⁺ and non-native metals into PPIX. The figure present progress spectra of Fe²⁺ insertion into PPIX by EcHemH. The black trace represents the UV-visible spectrum of 7.5 μM proto-porphyrin IX in reaction buffer (100 mM MOPS, 400 mM NaCl, 0.2% w/v Tween 80 at pH 7.0) with 100 nM EcHemH. The metalation reaction was initiated at 25° C. by the addition of 10 μM of Fe²⁺. To limit competing oxidation to Fe³⁺, samples were prepared fresh and initial velocities were taken from the first 30 s of data acquisition. The grey line traces indicate absorption spectra taken during the reaction progress. The broken line trace represents the absorption spectrum of heme b (“FePPIX”) at the end of the reaction.

FIG. 2B is a graph depicting initial rates of EcHemH-catalyzed heme production plotted as a function of the Fe²⁺ concentration. The concentration of PPIX was 5 μM. These reactions were conducted at 25° C. The solid line represents the best fit of the data using an ordered sequential binding kinetic model (data not shown).

FIG. 2C shows progress spectra of Co²⁺ insertion into PPIX by EcHemH. The experimental setup is identical to FIG. 2A, except that 30 nM EcHemH and 10 μM Co²⁺ were used in the reaction. The broken line trace represents the absorption spectrum of CoPPIX at the end of the reaction.

FIG. 2D is a graph showing the initial rates of EcHemH-catalyzed CoPPIX production plotted as a function of Co²⁺ concentration. Rates were measured with PPIX concentrations ranging from 0.1-10 μM PPIX, only three concentrations of which are shown here for clarity: 0.5 μM (▴), 1.0 μM (♦), and 5.0 μM (●). The data were fit globally using DynaFit 4 software (Biokin, Ltd., Watertown, Massachusetts).

FIG. 2E is a schematic representation of a proposed noncompetitive substrate inhibition kinetic scheme.

FIG. 2F shows progress curves tracking CuPPIX production over time. Reactions were conducted at 25° C. with approximately 5 μM (grey) or 10 μM (black) PPIX and 100 nM EcHemH and were initiated by the addition of approximately 3.8 μM Cu²⁺. Solid lines represent the best global fit of the data by DynaFit 4 to the noncompetitive substrate inhibition model and correspond the kinetic parameters shown in Table 1.

FIG. 3A is a structural model of EcHemH showing a selection of residues for site saturation mutagenesis.

FIG. 3B is a histogram showing results from a re-screen of “hits” from site-saturation mutagenesis of EcHemH in cell lysates. Each variant was screened in biological quadruplicate with 10 μM PPIX and a total of 1 mM divalent metal substrate. The resulting porphyrin distribution was analyzed by UPLC. Bars represent the average relative product peak areas corresponding to CoPPIX (solid grey), NiPPIX (diagonal hatching), and CuPPIX (horizontal hatching). Black dots represent the relative amount of PPIX substrate remaining, a measure of each variant's total activity. Parent EcHemH is indicated by black borders (fourth entry from left).

FIGS. 4A (in vivo) and 4B (in vitro) present the results for competition experiments for Co²⁺and Fe²⁺ incorporation into protoporphyrin IX (PPIX) by parent EcHemH and L13R EcHemH variant. FIG. 4A shows the distribution of MPPIX products obtained from co-expression of parent and L13R EcHemH with dye-decolorizing peroxidase (DyP) with a defined mixture of 50 μM Fe²⁺ and 100 μM Co²⁺ added to the growth media. DyP was purified by Ni-affinity chromatography and digested in nitric acid prior to analysis by ICP-MS. Data are average of triplicate ICP-MS measurements. FIG. 4B shows the in vitro measurement of Co²⁺ versus Fe²⁺ incorporation into PPIX by purified parent and L13R EcHemH enzymes. Reactions were conducted in 100 mM Tris-HCl buffer with 400 mM NaCl and 0.2% Tween 80. The concentration of enzyme added was 250 nM and the concentration of PPIX was 5.0 μM. Reactions were initiated with the addition of 50 μM each of Fe²⁺ and Co²⁺ in 0.5 mM ascorbate. After 20 minutes, reactions were quenched with acid and extracted into organic solvent. The relative concentrations of porphyrin products were analyzed by UPLC using the absorbance at 400 nm (FePPIX and PPIX) and 423 nm (CoP-PIX). Error bars represent the standard deviation of quadruplicate UPLC measurements.

FIGS. 5A, 5B, and 5C show the effect of HemH expression, cobalt concentration, and E. coli expression strain on the distribution of CoPPIX-and heme-loaded DyP. FIG. 5A shows metal content analysis of DyP by ICP-MS. Proteins were expressed in BL21(DE3) in the absence of a co-expression vector, with parent EcHemH, and with EcHemH L13R. CoCl₂ (1 mM) and 250 μM δ-aminolaevulinic acid were added at the time of induction. Dots represent biological replicates. FIG. 5B shows ICP-MS measurements of DyP expressed in BL21(DE3) with various amounts of cobalt. Cobalt was added when OD₆₀₀ reached 0.2-0.3, prior to induction with d-arabinose. Measurements were made in triplicates and error bars represent standard deviation. FIG. 5C shows ICP-MS measurements of DyP expressed in JM109(DE3) with parent and L13R EcHemH co-expression, and in the absence of chelatase co-expression. Measurements were made in triplicate and error bars represent standard deviations.

FIG. 6A shows the UV-visible spectra of as-purified CoDyP (solid line) and after reduction with sodium dithionite (broken line).

FIG. 6B is a histogram showing expression titers for CoPPIX (grey) and native heme (DyP; square hatching) in M9* medium plus cobalt, TB medium plus cobalt, and TB medium.

### DETAILED DESCRIPTION OF THE INVENTION

Disclosed herein is a method of making unnatural CoPPIX and NiPPIX in an E. coli strain. The method does not require genetic engineering, evolutionary adaptation, or auxiliary plasmids. Most notably, the method can be performed using rich media—that is, without the need for using iron-deficient growth media to grow the E. coli cells. Using the present method, the E. coli efficiently incorporate the cofactors into hemoproteins. The preferred cell line for use in the method is E. coli BL21(DE3). This E. coli strain is available from several commercial suppliers, including New England Biolabs, Fischer Scientific, and others. Other E. coli strains may also be used.

For purposes of brevity only, the description that follows is directed to the method of producing the unnatural cobalt-containing cofactor, CoPPIX. This is solely to avoid redundancy. The method disclosed herein functions in analogous fashion to produce the analogous NiPPIX cofactor.

Because the method uses a rich growth media, the E. coli thrives. This is in stark contrast to the prior art approaches, which require using an iron-deficient grown media. Such iron-deficient media literally starve the E. coli, leading to low titers and low production of the CoPPIX cofactor. In the present method, the E. coli is grown in rich media supplemented with a high concentration of cobalt (or nickel, or copper, or zinc for the corresponding analogs). The toxicity of free cobalt, which is present in high concentrations in the growth medium, is partly offset by overexpression of the hemoprotein. The enhanced cobalt tolerance of the cells is likely due to sequestration of toxic cobalt within the hemoprotein in the form of CoPPIX. Surprisingly, cells rendered tolerant to cobalt by serial passaging were less efficient at incorporating CoPPIX. Most surprisingly (and unexpectedly) it was found that selecting the cells for cobalt tolerance enhanced incorporation of the native FePPIX. When E. coli BL21(DE3) cells are grown in augmented minimal media supplemented with 500 μM CoCl₂, CoPPIX is readily biosynthesized and incorporated into a variety of heterologously expressed hemoproteins with an efficiency that is comparable to their native FePPIX cofactor. Further analysis showed that CoPPIX is incorporated into the native binding site in the expressed protein, with only trace amounts of FePPIX observed.

The utility of cobalt-substituted hemoproteins (as well as the nickel-, copper-, and zinc-containing analogs) is broad. CoPPIX is an excellent spectroscopic analog of heme that has been used extensively in EPR and resonance Raman studies to characterize hemoproteins. Notably, the study of synthetic cobalt porphyrin complexes suggests there is a wealth of chemistry that remains to be explored and expanded upon in a biocatalytic setting. See Davidson, et al. “Metal Ion Selectivity and Substrate Inhibition in the Metal Ion Chelation Catalyzed by Human Ferrochelatase,” J. Biol. Chem. 2009, 284 (49), 33795-33799; Hunter, et al. “Metal Ion Substrate Inhibition of Ferrochelatase,” J. Biol. Chem. 2008, 283 (35), 23685-23691; and Mcintyre, et al. “Nickel(II) Chelatase Variants Directly Evolved from Murine Ferrochelatase: Porphyrin Distortion and Kinetic Mechanism,” Biochemistry 2011, 50 (9), 1535-1544.

Cobalt itself is attractive as it is abundant and inexpensive compared to many transition metals. This route to CoPPIX synthesis and bio-incorporation leverages a common laboratory strain and requires no auxiliary plasmids. Protein expression using the present method does not require specialized equipment, anaerobic culture conditions, or supplementation of a pre-synthesized cofactor. Consequently, this method can be applied using common resources by any research group equipped for protein expression in E. coli. Further, the straightforward nature of this approach lends itself to directed evolution, which may unlock new modes of biocatalytic CoPPIX reactivity.

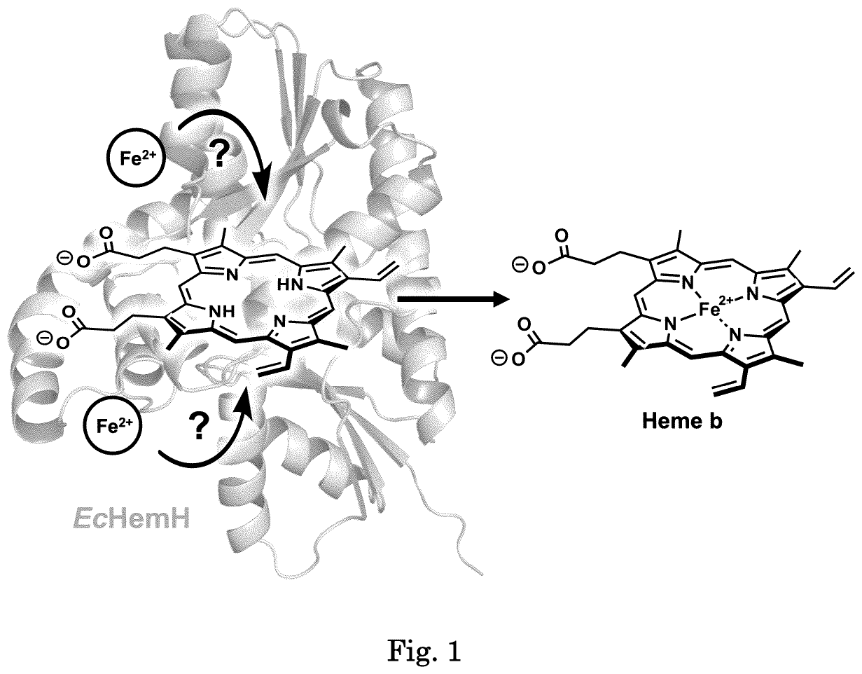

Metalation of tetrapyrrole-derived cofactors (heme, siroheme, cobalamin, and others) is a physiologically irreversible process catalyzed by a diverse class of enzymes called chelatases. In E. coli, the final step in heme b biosynthesis is incorporation of ferrous iron into protoporphyrin IX (PPIX), catalyzed by PPIX ferrochelatase (EcHemH). See FIG. 1. Eukaryotic HemH homologs, denoted FECH, particularly those from S. cerevisiae and H. sapiens, are well-studied. EcHemH is a membrane-associated enzyme and has been previously expressed and purified, but no kinetic characterization of EcHemH, or HemH from any other gram-negative bacteria, has been reported. The location of the productive metal binding site and the face of the porphyrin into which the metal is inserted have been debated in the literature. See Dailey, et al. “Ferrochelatase at the Millennium: Structures, Mechanisms and [2Fe-2S] Clusters,” Cell. Mol. Life Sci. 2000, 57 (13-14) and Hunter, et al. “Metal Ion Coordination Sites in Ferrochelatase,” Coord. Chem. Rev. 2022, 460, 214464. Nevertheless, in vitro incorporation of non-native metals by several ferrochelatases (HemH homologs) is well-known. To date, it is unclear how chelatases tune their activity for different metals and many potential effects may contribute depending on the specific protein and metal in question. Distortion of the porphyrin, specificity-determining active site residues, differential product inhibition, and exogenous metal delivery are suggested to contribute.

The method disclosed herein, however, takes advantage of E. coli BL21(DE3)'s heretofore unknown ability to produce CoPPIX in vivo. The native metal specificity of EcHemH, was investigated using purified enzyme to measure its catalytic activity with Fe²⁺, Co²⁺, Ni²⁺, Cu²⁺, and Zn²⁺. These investigations were undertaken with the thought that a more selective cobalt chelatase might enable efficient production and incorporation of CoPPIX in iron-rich growth media. A substrate-multiplexed metal specificity was used to identify a single point mutant (L13R) that has a ˜30-fold shift in selectivity for Co²⁺ over Fe²⁺ relative to the parent EcHemH. The ability of EcHemH variants were analyzed to arrive at a method that selectively produces CoPPIX over heme in rich growth media. It was found that, while variants may produce altered specificity profiles in vivo, metabolic factors play the predominant role in determining CoPPIX incorporation into co-expressed hemoproteins. Most surprisingly, it was found that under sufficiently high cobalt concentrations in rich media and in the absence of a specifically engineered chelatase, BL21(DE3) reproducibly yields greater than 95% cobalt-loaded hemoprotein with titers comparable to typical hemoprotein expressions. The results disclosed herein provide insight into the mechanism and metal selectivity of EcHemH, inform future engineering efforts for metalloporphryin biosynthesis, and demonstrate a straightforward and scalable route to cobalt-substituted hemo-protein production in rich media.

### In Vitro Assessment of E. coli Ferrochelatase Metal Promiscuity

The effort began by probing the kinetic mechanism and metal specificity of EcHemH (UniProt accession: A0A140NEM8). This protein contains a C-terminal His-tag and was over-expressed in E. coli BL21(DE3), followed by purification with nickel affinity chromatography yielding approximately 10 mg EcHemH per L culture. While poly-His tags may interfere with the metal binding properties of some enzymes, such constructs for chelatase homologs have been used previously, without complication. See, for example, Brindley, et al. “A Story of Chelatase Evolution: Identification and Characterization of a Small 13-15-KDa “Ancestral” Cobaltochelatase (CbiXs) in the Archaea,” J. Biol. Chem. 2003, 278 (25), 22388-22395. Sodium cholate was added to the lysis, purification, and storage buffers, as the enzyme has been shown to require detergents for solubility. EcHemH is predicted to be membrane associated due to the presence of a 12-residue segment implicated in membrane association. Dailey, et al. “Ferrochelatase at the Millennium: Structures, Mechanisms and [2Fe-2S] Clusters,” Cell. Mol. Life Sci. 2000, 57 (13), 1909-1926.

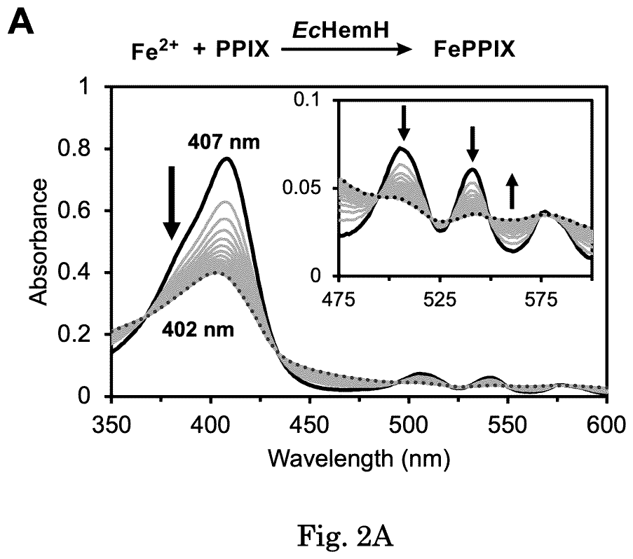

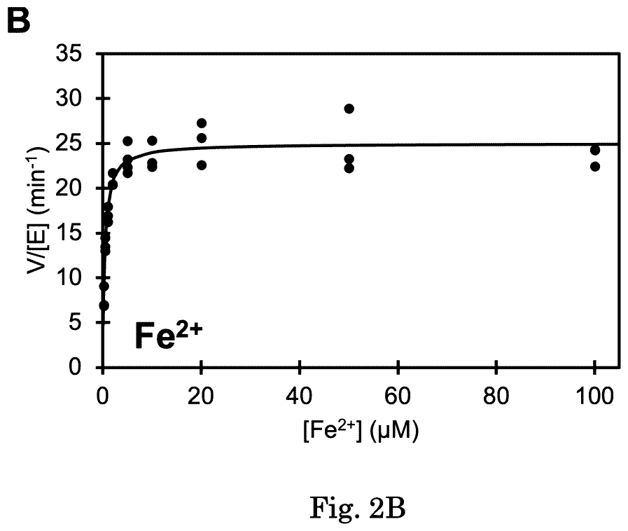

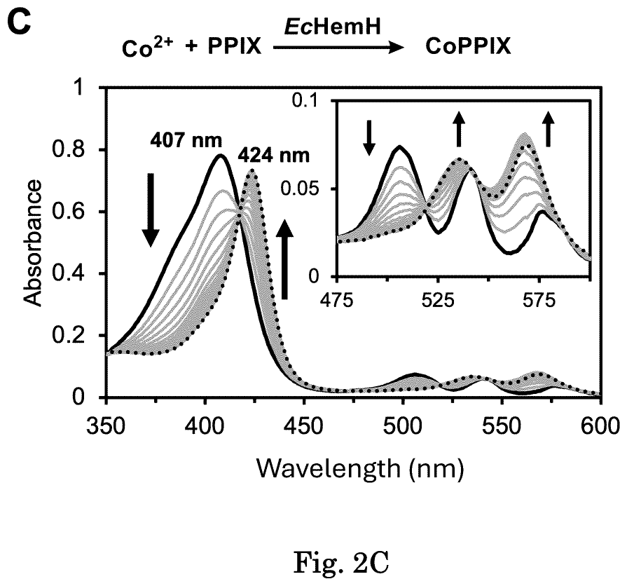

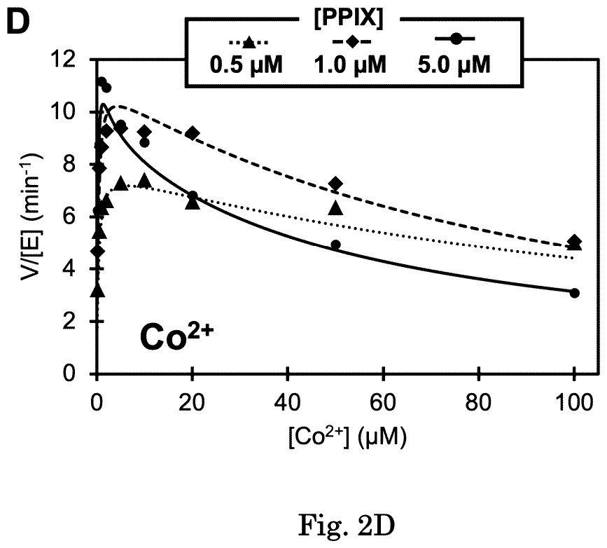

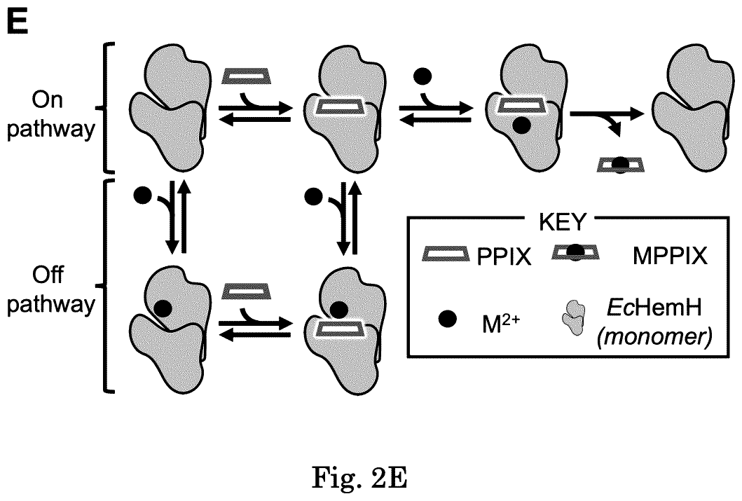

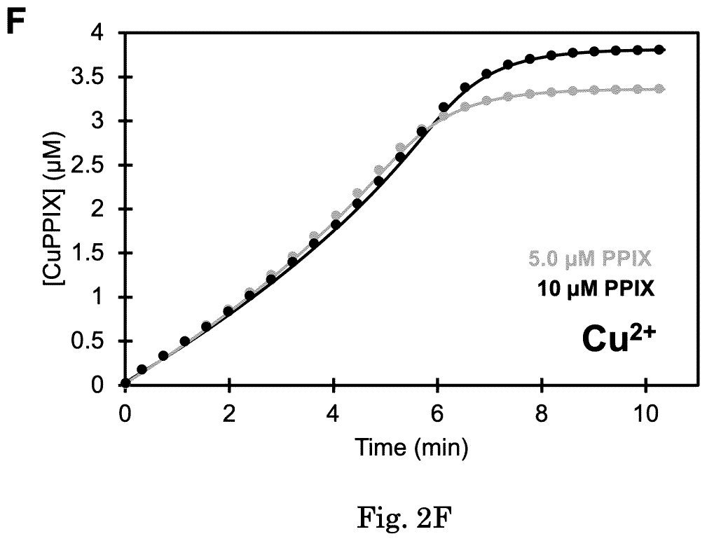

Using a simple spectroscopic assay, the ability of EcHemH to catalyze insertion of Fe²⁺, Co²⁺, Ni²⁺, Zn²⁺, Cu²⁺, Mn²⁺, and Mg²⁺ into PPIX was investigated. The catalytic activity of ferrochelatase is well-suited to kinetic analysis by electronic-absorption spectroscopy because the metalation of PPIX imparts unique spectral shifts to the Soret (˜400 nm) and Q bands (˜500-600 nm). Product formation for all metals tested was discovered except for Mn²⁺ and Mg²⁺ (See. FIGS. 2A and 2C). The reactivity profile of EcHemH was in good agreement with reported activity of previously characterized ferrochelatase enzymes. To quantify the native iron chelatase activity of EcHemH, the initial rates of heme formation were measured spectrophotometrically with variable PPIX and Fe²⁺ concentrations at room temperature (FIG. 2A). These data were fit to an ordered sequential mechanism where PPIX binds first, followed by Fe²⁺ binding. An equilibrium random-ordered mechanism can also describe these data, which results in identical macroscopic constants as the ordered sequential kinetic model. The resulting fit yielded a k_{cat} of 30 min⁻¹, K_{M}^{Fe} of 0.48 μM Fe²⁺, and K_{M}^{PPIX} of 1.1 μM PPIX. (Data not shown.) These values compare favorably with previously characterized ferrocheletase enzymes. Hunter, et al. “Metal Ion Substrate Inhibition of Ferrochelatase,” J. Biol. Chem. 2008, 283 (35), 23685-23691.

The kinetics of CoPPIX formation by EcHemH was then evaluated. The initial rates of CoPPIX production at 5 μM PPIX were measured while varying the Co²⁺ concentration. In contrast to the behavior observed with Fe²⁺, a decrease in the rate of CoPPIX production was seen with high concentrations of Co²⁺ (FIG. 2D). This substrate inhibition has been observed previously with yeast and mouse FECH enzymes, as well as with coproporphyrin(III) ferrochelatase (CpfC) of other species. To characterize this inhibition further, the initial rates of CoPPIX production were measured while varying the concentrations of both Co²⁺ and PPIX (FIG. 2D, 3 of 6 PPIX concentrations shown for clarity). The resulting data show that increasing concentrations of PPIX exacerbate the inhibitory effect of Co²⁺. These data mirror observations by Davidson, et al. in studies of human HemH with a PPIX analog, mesoporphyrin IX, and Zn²⁺. Davidson, et al. “Metal Ion Selectivity and Substrate Inhibition in the Metal Ion Chelation Catalyzed by Human Ferrochelatase,” J. Biol. Chem. 2009, 284 (49), 33795-33799.

To better understand the nature of Co²⁺ inhibition, a global analysis of the Co²⁺ initial rate data was performed to identify the simplest kinetic mechanism that recapitulates this complex kinetic behavior. Notably, none of the existing kinetic models for chelatases in the literature fit the data. (Data not shown.) On the basis of this analysis, a possible EcHemH kinetic mechanism that includes two kinetically distinct metal binding modes, only one of which is kinetically productive is shown in FIG. 2E. The productive pathway is initiated by PPIX binding. Co²⁺ can then bind in either a productive mode, leading to metal insertion, or a non-productive mode corresponding to noncompetitive inhibition. In this model, metal can also bind prior to PPIX, but leads to an inhibitory ternary complex. Together, these model features accurately recapitulate key kinetic observations. At low Co²⁺ concentrations, metal binding in the catalytically productive mode (i.e. after PPIX binding) dominates, and the initial velocities increase as the concentration of PPIX increases. However, at high PPIX concentrations, increasing Co²⁺ concentrations slow the metalation because noncompetitive binding occurs, leading to pronounced substrate inhibition (FIG. 2E). The Fe²⁺ insertion does not show substrate inhibition within the range of concentrations tested here, nor has substrate inhibition with Fe²⁺ been observed with other ferrochelatases. However, substrate inhibition was previously observed for the Cu²⁺ insertion into PPIX by the yeast homolog. Hunter, et al. “Metal Ion Substrate Inhibition of Ferrochelatase,” J. Biol. Chem. 2008, 283 (35), 23685-23691. These studies showed a distinctive ‘S’ shaped progress curves wherein reactions accelerate during a time course due to relief of substrate inhibition. CuPPIX formation by EcHemH was tracked spectroscopically with three different PPIX and Cu²⁺ concentrations and observed the same S-shape in the progress curves (FIG. 2F). These progress curve data were fit to the noncompetitive binding model (FIG. 2E); the data were well recapitulated, including the characteristic S-feature. The macroscopic kinetic parameters for insertion of Fe²⁺, Co²⁺, and Cu²⁺, were compared, which affirms the remarkable promiscuity of the enzyme. See Table 1.

TABLE 1
Kinetic profile for EcHemH activity with various metals
Macroscopic kinetic parameters

| Substrate | k_{cat} (min⁻¹) | K_{M} (metal) (μM) | k_{cat}/K_{M} (μM⁻¹ min⁻¹) |
|---|---|---|---|
| Fe²⁺ | 30 | 0.48 | 63 |
| Co²⁺ | 54 | 1.1 | 48 |
| Cu²⁺ | 34 | 0.94 | 36 |

Highest observed rates for metal insertion

| Substrate | V₀/[E] (min⁻¹) | [PPIX] (μM) | [M²⁺] (μM) |
|---|---|---|---|
| Fe²⁺ | 29 | 5 | 10 |
| Co²⁺ | 11 | 5 | 1 |
| Ni²⁺ | 6.8 | 2 | 200 |
| Zn²⁺ | 17 | 5 | 0.5 |

In Table 1, apparent macroscopic kinetic parameters for Fe²⁺, Co²⁺, and Cu²⁺ were derived from fitting the data to the appropriate kinetic model. Parameters were not well determined for Zn²⁺ or Ni²⁺. Instead, the highest observed initial rate is given for these metals, along with the corresponding metal and protoporphyrin IX (PPIX) concentrations.

EcHemH activity with Ni²⁺ and Zn²⁺ was also tested. The initial rates of NiPPIX production were measured at multiple concentrations of PPIX and Ni²⁺. Like Co²⁺, these data showed substrate inhibition by the metal that became more pronounced as a function of PPIX. (Data not shown). While the proposed mechanism fit these data well, the model was underdetermined, and macroscopic rate constants could not be confidently assigned. Analysis of ZnPPIX formation also showed distinctive kinetic behaviors. These data showed the fastest initial velocity of metal insertion among the various metals tested, but also the strongest substrate inhibition. These features were recapitulated by the noncompetitive substrate inhibition model, but the model was again underdetermined. Other kinetic possibilities for Ni²⁺ or Zn²⁺ based on these data alone cannot be ruled out and it is possible that different metals engage in distinct kinetic mechanisms. Nevertheless, a heuristic comparison of activity on the various metals highlights the prevalence of substrate inhibition with non-native metals and the relative efficiency of EcHemH with diversity of transition metals.

### Bioinformatic Analysis of EcHemH Sequence and Structure

It was hypothesized that the promiscuous EcHemH activity with Co²⁺ was the dominant factor in enabling CoPPIX production in BL21(DE3). If true, selective CoPPIX production in iron-rich media might be enabled by altering the specificity of the chelatase via protein engineering to favor Co²⁺ over Fe²⁺. The structural underpinnings of metal specificity in ferrochelatase homologs are not well understood, although examples of variants with altered specificity have been found. Mcintyre, et al. “Nickel(II) Chelatase Variants Directly Evolved from Murine Ferrochelatase: Porphyrin Distortion and Kinetic Mechanism,” Biochemistry 2011, 50 (9), 1535-1544.

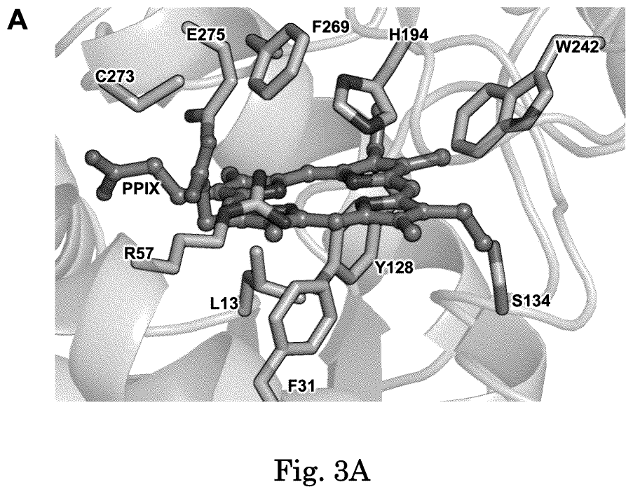

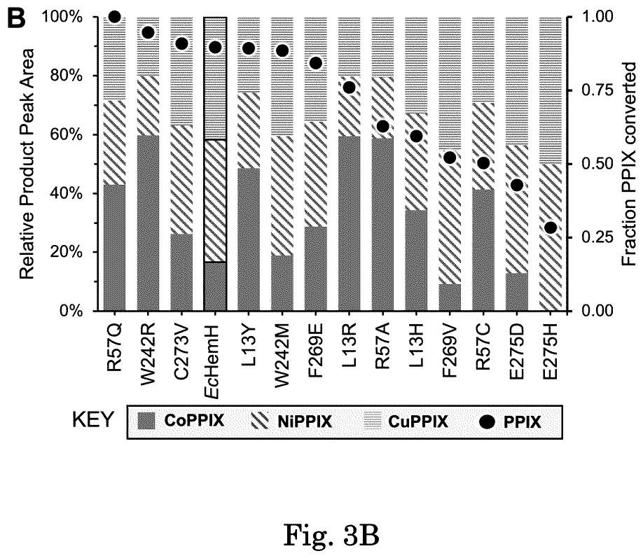

A model generated with AlphaFold software (European Molecular Biology Laboratory—European Bioinformatics Institute, Cambridge, England) was used to compare structural features of EcHemH to other, structurally characterized homologs. See FIG. 3A. Alignment of EcHemH with the PPIX-bound HsFECH structure (PDB ID: 2HRE; 27% sequence identity) suggested how EcHemH might interact with the porphyrin substrate. The main structural difference between HsFECH and EcHemH is the presence of an iron-sulfur cluster at the C-terminus of HsFECH. Despite the low overall sequence identity, many active site residues appear highly conserved. Distinctive residues in EcHemH are Leu13, Phe31, and Cys273, which correspond to Met76, Leu92, and His341 in HsFECH, respectively (FIG. 3B). The differences at Leu13 (E. coli) and Met76 (human) are noteworthy because these residues lie directly below the porphyrin substrate and Met76 has been implicated in metal specificity for HsFECH. Dailey, et al. “Ferrochelatase at the Millennium: Structures, Mechanisms and [2Fe-2S] Clusters,” Cell. Mol. Life Sci. 2000, 57 (13), 1909-1926.

To gain further insight into sequence conservation across the enzyme family, a sequence alignment of 5,026 HemH homologs was generated which showed active site conservation with logo plots. (Data not shown.) Several striking patterns were observed. For example, His194, Trp242, Phe269, and Glu275 (E. coli numbering) are >99% conserved, suggesting that these residues are especially important for ferrochelatase function. Additionally, the positions corresponding to Leu13 and Phe31 are almost exclusively occupied by hydrophobic residues. This analysis provides evolutionary context for choosing which residues might be most amendable to mutation. Site saturation mutagenesis (SSM) libraries at 10 active site residues were screened. See FIG. 3A, labelled residues. Based on the structural model, it was hypothesized these residues might impart changes in metal specificity either by altering metal chelation or by altering the extent and nature of the porphyrin distortion.

The primers used to amplify EcHemH and to generate the site saturation library are presented in Table 2:

TABLE 2
Primers for amplifying EcHemH and generating site-saturation libraries. Entries
where three primers are listed denotes a 12:9:1 mixture of primers shown
(bearing NDT, VHG, TTG degenerate codons, in bold, respectively), for a total of
22-codons represented in the resulting libraries.

| Name | Sequence |
|---|---|
| HemH fw | GTTTAACTTTAAGAAGGAGATATACATATGCGTCAGACTAAAACCGGTATC |
| | (SEQ. ID. NO: 1) |
| HemH rv | CCGGATCTCAATGGTGATGGTGATGGTGGCGATACGCGGCAACAAGATTAG |
| | (SEQ. ID. NO: 2) |
| L13X fw | GTATCCTGCTGGCAAACNDTGGTACGCCCGATGC (SEQ. ID. NO: 3) |
| | GTATCCTGCTGGCAAACVHGGGTACGCCCGATGC (SEQ. ID. NO: 4) |
| | GTATCCTGCTGGCAAACTTGGGTACGCCCGATGC (SEQ. ID. NO: 5) |
| L13X rv | GTTTGCCAGCAGGATACCGGTTTTAGTCTGAC (SEQ. ID. NO: 6) |
| F31X fw | GGTAAAACGCTATCTGAAACAANDTTTAAGCGACAGACGC (SEQ. ID. NO: 7) |
| | GGTAAAACGCTATCTGAAACAAVHGTTAAGCGACAGACGC (SEQ. ID. NO: 8) |
| | GGTAAAACGCTATCTGAAACAATTGTTAAGCGACAGACGC (SEQ. ID. NO: 9) |
| F31X rv | TTGTTTCAGATAGCGTTTTACCGCTTCAGGTGTG (SEQ. ID. NO: 10) |
| R57X fw | CGTGATTTTGCCGCTGNDTTCGCCGCGTGTGGC (SEQ. ID. NO: 11) |
| | CGTGATTTTGCCGCTGVHGTCGCCGCGTGTGGC (SEQ. ID. NO: 12) |
| | CGTGATTTTGCCGCTGTTGCGCCGCGTGTGGC (SEQ. ID. NO: 13) |
| R57X rv | GCGGCAAAATCACGCCGCGCAGCAATGGCC (SEQ. ID. NO: 14) |
| Y128X fw | GTGGTGCTGCCGCTTNDTCCGCAATACTCC (SEQ. ID. NO: 15) |
| | GTGGTGCTGCCGCTTVHGCCGCAATACTCC (SEQ. ID. NO: 16) |
| | GTGGTGCTGCCGCTTTTGCCGCAATACTCC (SEQ. ID. NO: 17) |
| Y128X rv | GCGGCAGCACCACAATATGATCTACATGC (SEQ. ID. NO: 18) |
| S134X fw | GTGCTGCCGCTTTATCCGCAATACTCCTGTNDTACGGTCGGTGC (SEQ. ID. NO: 19) |
| | GTGCTGCCGCTTTATCCGCAATACTCCTGTVHGACGGTCGGTGC (SEQ. ID. NO: 20) |
| | GTGCTGCCGCTTTATCCGCAATACTCCTGTTTGACGGTCGGTGC (SEQ. ID. NO: 21) |
| S134X rv | GGATAAAGCGGCAGCACCACAATATGATCTACATGCTCTGCCAG (SEQ. ID. NO: 22) |
| H194X fw | CTGCTGCTGCTCTCTNDTTGGGGCATTCCCCAGCG (SEQ. ID. NO: 23) |
| | CTGCTGCTGCTCTCTVHGTGGGGCATTCCCCAGCG (SEQ. ID. NO: 24) |
| | CTGCTGCTGCTCTCTTTGTGGGGCATTCCCCAGCG (SEQ. ID. NO: 25) |
| H194X rv | TAAGAGAGCAGCAGCAGATCCGGTTCG (SEQ. ID. NO: 26) |
| W242X fw | CGCTTTGGTCGGGAACCCNDTCTGATGCCTTATACCG (SEQ. ID. NO: 27) |
| | CGCTTTGGTCGGGAACCCVHGCTGATGCCTTATACCG (SEQ. ID. NO: 28) |
| | CGCTTTGGTCGGGAACCCTTGCTGATGCCTTATACCG (SEQ. ID. NO: 29) |
| W242X rv | GGTTCCCGACCAAAGCGCGACTGAAAGGTCATCATCAC (SEQ. ID. NO: 30) |
| F269X fw | GGTGATGTGCCCGGGCNDTGCTGCGGATTG (SEQ. ID. NO: 31) |
| | GGTGATGTGCCCGGGCVHGGCTGCGGATTG (SEQ. ID. NO: 32) |
| | GGTGATGTGCCCGGGCTTGGCTGCGGATTG (SEQ. ID. NO: 33) |
| F269X rv | GCCCGGGCACATCACCTGTATATGAC (SEQ. ID. NO: 34) |
| C273X fw | GCTTTGCTGCGGATNDTCTGGAGACGCTG (SEQ. ID. NO: 35) |
| | GCTTTGCTGCGGATVHGCTGGAGACGCTG (SEQ. ID. NO: 36) |
| | GCTTTGCTGCGGATTTGCTGGAGACGCTG (SEQ. ID. NO: 37) |
| C273X rv | ATCCGCAGCAAAGCCCGGGCAC (SEQ. ID. NO: 38) |
| E275X fw | GCTGCGGATTGTCTGNDTACGCTGGAAGAGATTGC (SEQ. ID. NO: 39) |
| | GCTGCGGATTGTCTGVHGACGCTGGAAGAGATTGC (SEQ. ID. NO: 40) |
| | GCTGCGGATTGTCTGTTGACGCTGGAAGAGATTGC (SEQ. ID. NO: 41) |
| E275X rv | CAGACAATCCGCAGCAAAGCCCGG (SEQ. ID. NO: 42) |

TABLE 3
Gene sequences used. Start/Stop codon bolded.

| Name | Sequence |
|---|---|
| EcHemH | ATGCGTCAGACTAAAACCGGTATCCTGCTGGCAAACCTGGGTACGCCCGATGCCCCCA |
| | CACCTGAAGCGGTAAAACGCTATCTGAAACAATTTTTAAGCGACAGACGCGTGGTTGA |
| | TACCTCACGGTTGTTATGGTGGCCATTGCTGCGCGGCGTGATTTTGCCGCTGCGCTCG |
| | CCGCGTGTGGCGAAGCTGTATGCCTCTGTCTGGATGGAAGGTGGCTCGCCGCTGATGG |
| | TTTACAGCCGTCAGCAACAGCAGGCGCTGGCACAACGTTTACCGGAGACGCCCGTAGC |
| | GCTGGGAATGAGCTACGGCTCGCCATCACTGGAAAGCGCCGTAGATGAACTCCTGGCA |
| | GAGCATGTAGATCATATTGTGGTGCTGCCGCTTTATCCGCAATACTCCTGTTCAACGG |
| | TCGGTGCGGTATGGGATGAACTGGCACGCATTCTGGCGCGCAAACGTAGCATTCCGGG |
| | GATATCGTTTATTCGTGATTACGCTGATAACCACGATTACATTAATGCACTGGCGAAC |
| | AGCGTACGCGCTTCTTTTGCCAAACATGGCGAACCGGATCTGCTGCTGCTCTCTTATC |
| | ATGGCATTCCCCAGCGTTATGCAGATGAAGGCGATGATTACCCGCAACGTTGCCGCAC |
| | AACGACTCGCGAACTGGCTTCCGCACTGGGGATGGCACCGGAAAAAGTGATGATGACC |
| | TTTCAGTCGCGCTTTGGTCGGGAACCCTGGCTGATGCCTTATACCGACGAAACGCTGA |
| | AAATGCTCGGAGAAAAAGGCGTAGGTCATATACAGGTGATGTGCCCGGGCTTTGCTGC |
| | GGATTGTCTGGAGACGCTGGAAGAGATTGCCGAGCAAAACCGTGAGGTCTTCCTCGGT |
| | GCCGGCGGGAAAAAATATGAATATATTCCAGCGCTTAATGCCACGCCGGAACATATTG |
| | AAATGATGGCTAATCTTGTTGCCGCGTATCGCCACCATCACCATCACCATTGA |
| | (SEQ. ID. NO: 43) |
| DyP | ATGTCACAAGTGCAGTCTGGCATTCTTCCAGAGCACTGTCGTGCAGCCATCTGGATCG |
| | AGGCAAACGTGAAAGGGGATGTCAATGCACTGCGCGAGTGCTCAAAAGTCTTTGCAGA |
| | TAAATTAGCTGGTTTCGAGGCACAGTTTCCAGACGCACATCTTGGAGCGGTCGTTGCC |
| | TTTGGACATGACACATGGCGTGCTTTGTCGGGGGGGGTAGGGGCCGAGGAATTGAAGG |
| | ATTTCACGCCTTATGGCAAGGGTTTGGCTCCAGCCACTCAGTACGATGTCCTTATCCA |
| | TATTTTATCTCTGCGTCACGACGTAAATTTTTCCGTCGCCCAAGCGGCCATGGCCGCT |
| | TTTGGGGATGCTGTGGAGGTGAAAGAAGAGATTCACGGCTTTCGCTGGGTCGAAGAAC |
| | GTGACTTATCTGGGTTCGTAGATGGGACAGAAAATCCAGCGGGGGAAGAAACACGCCG |
| | TGAAGTCGCCGTTATTAAAGATGGGGTTGACGCCGGAGGCAGTTACGTTTTTGTGCAA |
| | CGCTGGGAGCATAACTTAAAACAACTTAACCGTATGTCAGTTCACGATCAGGAAATGA |
| | TGATCGGTCGTACAAAAGTTGCAAACGAAGAAATTGATGGCGATGAGCGCCCAGAGAC |
| | AAGCCACTTAACGCGTGTCGACCTTAAAGAGAACGGAAAAGGCTTAAAAATTGTTCGT |
| | CAGAGCTTGCCGTACGGTACTGCCAGCGGCACGCATGGATTGTATTTCTGCGCGTATT |
| | GCGCGCGCTTGTATAATATCGAGCAGCAGTTGCTGTCCATGTTCGGTGACACGGACGG |
| | AAAACGTGATGCAATGCTTCGCTTCACCAAGCCTGTTACAGGGGGTTACTACTTTGCG |
| | CCGTCGTTAGATAAATTACTTGCCTTGCTCGAGCACCATCACCATCACCATTGA |
| | (SEQ. ID. NO: 44) |
| CYP119 | ATGTACGATTGGTTTTCGGAGATGCGTAAAAAAGATCCTGTTTACTACGATGGTAATA |
| | TTTGGCAAGTATTTTCATATCGCTACACCAAGGAGGTATTAAACAATTTTTCAAAATT |
| | TTCTTCCGATTTAACAGGGTACCACGAGCGCCTTGAGGACTTGCGCAATGGAAAGATC |
| | CGCTTTGACATCCCAACCCGCTATACCATGCTTACGTCGGATCCGCCGCTGCATGACG |
| | AATTGCGTAGTATGAGTGCCGATATCTTTTCTCCGCAAAAGTTGCAAACCCTGGAAAC |
| | TTTTATTCGCGAAACGACCCGTAGTTTATTGGACTCGATTGACCCTCGCGAGGACGAT |
| | ATTGTTAAGAAGCTGGCCGTGCCCCTTCCAATTATCGTGATTTCCAAAATCCTGGGTC |
| | TGCCAATCGAGGACAAAGAGAAGTTCAAGGAATGGAGCGACCTTGTAGCGTTTCGTCT |
| | GGGCAAACCTGGCGAGATCTTCGAATTAGGAAAGAAGTATCTGGAATTGATCGGATAT |
| | GTGAAAGACCATCTTAATTCAGGTACGGAAGTGGTCAGCCGCGTAGTTAATTCGAATC |
| | TTTCTGACATTGAAAAATTAGGATATATCATTTTATTATTGATTGCCGGGAACGAGAC |
| | GACTACTAATCTGATTAGCAATTCAGTTATTGACTTCACGCGCTTCAACTTGTGGCAA |
| | CGCATTCGCGAAGAAAACCTTTACCTGAAAGCTATTGAAGAGGCTCTTCGCTATTCAC |
| | CCCCGGTTATGCGCACAGTTCGTAAAACCAAGGAGCGTGTAAAGTTGGGCGACCAGAC |
| | GATTGAGGAAGGAGAATACGTGCGCGTCTGGATTGCATCAGCGAACCGCGACGAAGAG |
| | GTCTTTCACGACGGAGAAAAGTTCATTCCAGATCGTAACCCGAATCCTCATCTGAGTT |
| | TCGGGAGTGGGATCCACCTGTGCCTGGGGGCACCTTTGGCACGTTTGGAAGCCCGTAT |
| | TGCCATCGAGGAGTTCTCGAAGCGTTTTCGTCACATCGAAATTCTTGACACAGAAAAG |
| | GTGCCAAACGAAGTGTTGAACGGGTATAAGCGTTTGGTAGTTCGTCTGAAGAGCAATG |
| | AGCTCGAGCACCATCACCATCACCATTGA (SEQ. ID. NO: 45) |

### Altering Metal Specificity of EcHemH Through Site-Saturation Mutagenesis

Substrate specificity can be a challenging feature to optimize through engineering because traditional approaches typically monitor activity on a single, model substrate as a proxy for overall enzyme performance.

These assays cannot distinguish between enzymes that have higher expression or higher catalytic activity. Here, there was more interest in the relative activity of variants on different metals in competition than in the overall catalytic prowess. Therefore, a substrate-multiplexed screen (SUMS) was developed with a mixture of metal ions, which provided direct information on enzyme specificity in a single experiment. The SUMS approach, as applied to protein engineering, has been shown to facilitate discovery of enzyme variants with altered specificity and identification of distal residues that impact catalysis. See, for example, Weeks, et al. “Engineering Peptide Ligase Specificity by Proteomic Identification of Ligation Sites,” Nat. Chem. Biol. 2018, 14 (1), 50-57.

While relative activity of Fe²⁺ versus Co²⁺ was the principal interest, it was found that the specificity between these two metals was exceedingly difficult to monitor in cell lysates. At least two factors confounded analysis. First, Fe²⁺ quickly oxidizes in lysate to Fe³⁺, which is not a substrate for EcHemH. Second, heme b is catabolized in cell lysate, leading to inconsistent ratios relative to CoPPIX. To work around these challenges, it was hypothesized that screening on a mixture of non-native metals would reveal residues that provide the most significant changes to metal specificity relative to the parent. These mutations could subsequently be analyzed to gain insight into their Fe²⁺ specificity using an alternative screening method.

Cell lysates containing EcHemH variants were added to a mixture of Co²⁺, Ni²⁺, and Cu²⁺ at relatively high concentration (1 mM total M²⁺) in the presence of 10 μM PPIX. The relative distribution of the porphyrin products was directly measured using ultra-pressure liquid chromatography (UPLC) and the concentration of each metal was adjusted such that the parent EcHemH produced approximately equal signal for CoPPIX, NiPPIX, and CuP-PIX. The uncertainties associated with screening preclude quantitative analysis on the absolute abundance of each product.

While site-saturation studies of HemH homologs have not been previously reported, the data here were in good general agreement with the detailed analysis performed on point mutants of homologous enzymes. For example, the EcHemH H194X library yielded no active variants (data not shown). Studies of corresponding variants of FECH (H263C and H263A HsFECH and H235L ScFECH) indicated these variants were inactive or had only trace activity. Indeed, H194 is nearly 100% conserved among HemH/FECH homologs, consistent with an essential catalytic role. See Sellers, et al. “Human Ferrochelatase: Characterization of Substrate-Iron Binding and Proton-Abstracting Residues,” Biochemistry 2001, 40 (33), 9821-9827 and Medlock, et al. “A π-Helix Switch Selective for Porphyrin Deprotonation and Product Release in Human Ferrochelatase,” J. Mol. Biol. 2007, 373 (4), 1006-1016.

To compare specificity shifts among variants from different libraries, a subset of variants that showed the largest shifts in specificity in the initial screen were re-screened in parallel. While the trends in activity were generally reproducible, several variants had different specificities in this follow-up assessment when compared to the initial screens. From the re-screen data, several variants were identified with altered metal specificity compared to the parent EcHemH that was chosen for further validation in comparison against Fe²⁺. See FIG. 3B, which presents the yields for CoPPIX, NiPPIX, CuPPIX, and native PPIX (i.e., FePPIX).

### Assessing EcHemH Variant Specificity for Fe²⁺ Versus Co²⁺

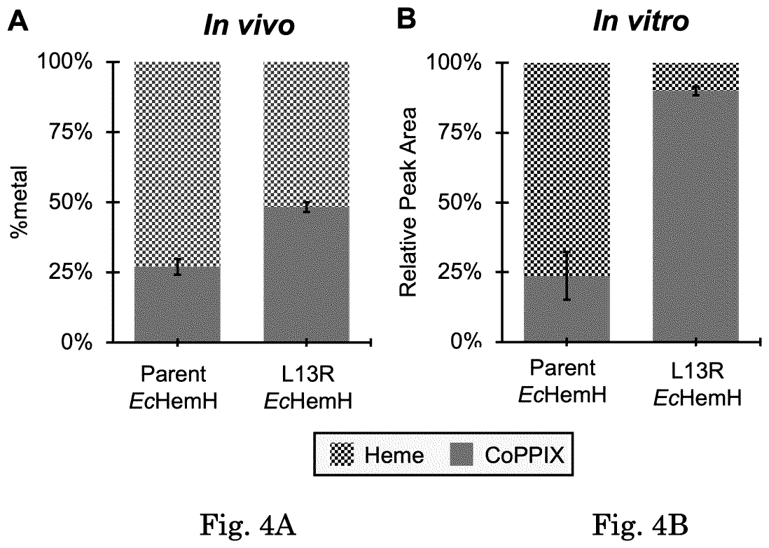

Variants with in vitro changes in metal specificity were then evaluated to see how they altered metalloporphyrin incorporation into hemoproteins in vivo. Cells harboring over-expressed EcHemH variants were grown in rich media supplemented with a 4:1 ratio of exogenous Co²⁺ to Fe²⁺, such that the bioavailable metal pool was consistent and well-defined. To directly assess the effect of chelatase identity on hemoprotein loading, EcHemH was co-expressed with the hemoprotein dye-decolorizing peroxidase (“DyP,” UniProt Accession E3G9I4). DyP binds free PPIX, CoPPIX, and heme b promiscuously. Following expression, DyP was purified by nickel-affinity chromatography and the relative Co²⁺ and Fe²⁺ content of the protein samples was measured using inductively coupled plasma mass spectrometry (ICP-MS). This analysis showed several variants incorporated more CoPPIX relative to the parent EcHemH, including L13R, L13H, R57Q, and E275D (FIG. 4A). Many factors, such as enzyme expression level, may impact metalation outcomes. Indeed, these in vivo experiments relied on overexpression of EcHemH and its variants to circumvent the native EcHemH regulation and deliver high concentrations of enzyme. However, there remained the possibility that high titers of EcHemH may influence metalloporphyrin production relative to the native BL21 (DE3) system used. DyP was therefore expressed and purified without overexpressing EcHemH. The metal content of the DyP was then determined. This experiment was intended as a negative control, but instead showed that the CoPPIX content of DyP without overexpression of EcHemH was the same or higher than any other conditions tested. This result was the first clue that ferrochelatase specificity may be a minor contributor to in vivo metalation outcomes. To rigorously test this new hypothesis, a single variant with altered specificity was validated and investigated for its impact (or lack thereof) on in vivo PPIX metalation.

### Testing the Effects of Altered Chelatase Specificity

EcHemH L13R was selected to assess the change in metal specificity relative to parent. Rather than determine complex kinetic parameters with this enzyme, a direct measurement of the change in selectivity was performed by assaying the metalation outcome when Co²⁺ and Fe²⁺ are in direct competition. Excess ascorbate was added to maintain the ferrous oxidation state; product formation was analyzed by UPLC. This analysis revealed that the L13R mutation imparts a ˜30-fold shift in selectivity for Co²⁺ relative to parent under these conditions. See FIG. 4B.

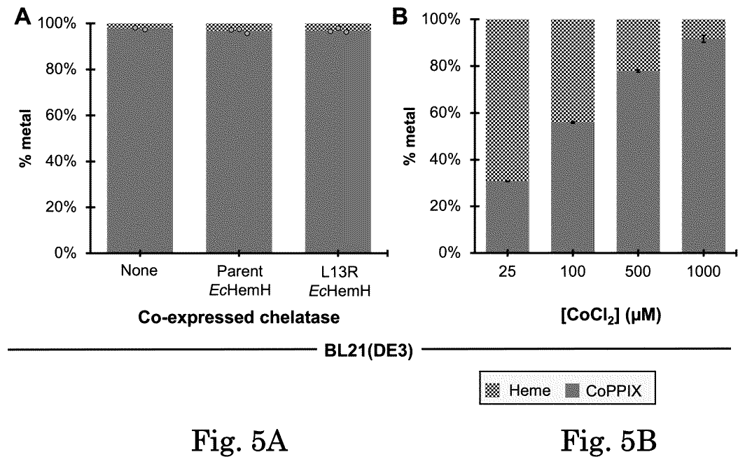

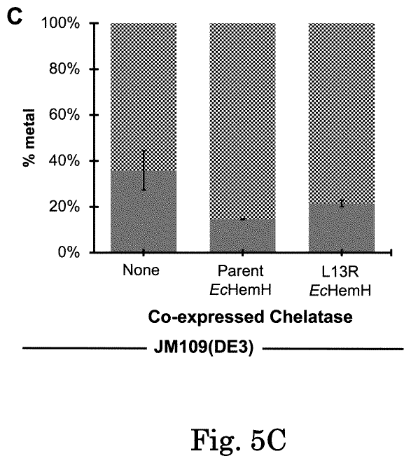

Next, the cobalt selective EcHemH variant L13R was co-expressed with the native EcHemH to see if it had any significant impact on CoPPIX incorporation into DyP versus heme incorporation when the E. coli were incubated in rich media. The effect of co-expression of parent EcHemH versus the L13R variant on the production of DyP was comparted at substantially higher concentrations of cobalt (500 μM) in rich media without added iron. These results were further compared to DyP expressed in unmodified BL21(DE3), that only contained natively expressed EcHemH. Completely unexpectedly, the resulting DyP hemoprotein samples contained greater than 95% CoPPIX in all cases, even the intended negative control with no co-expression of a ferrochelatase. See FIG. 5A. These results refuted the initial hypothesis that alterations to chelatase specificity would lead to more efficient CoPPIX incorporation into hemoproteins in vivo.

### Effect of Cobalt Concentration, E. coli Strain, and Metal Identity on Metalloporphyrin Production

The influence of cobalt concentration in rich media (Terrific Broth) on the metalloporphyrin content of expressed hemoprotein was investigated. Surprisingly and unexpectedly, adding 1 mM of Co²⁺ yielded >90% cobalt incorporation in rich media. See FIG. 5B. Although the wet cell mass derived from these expressions decreased as more cobalt was added, the holoprotein titers per liter cell culture were only slightly diminished (data not shown). Based on the earlier observations in minimal media, it was hypothesized that the cytotoxic effect of high intracellular cobalt concentrations may be mitigated by the expression of the CoPPIX-containing hemoprotein, which serves as a sink for excess, toxic Co²⁺.

Because these results were so surprising, the next step was to determine whether CoPPIX production ability was unique to the E. coli BL21(DE3) strain. Expression experiments were carried out with with JM109 (DE3), a K12-derived strain whose metal homeostasis is well-studied. Foster, et al. “Metalation Calculators for E. coli Strain JM109(DE3): Aerobic, Anaerobic, and Hydrogen Peroxide Exposed Cells Cultured in LB Media,” Metallomics 2022, 14 (9). While overall CoPPIX production was less efficient in this strain, the strain still produced almost 40% CoPPIX under the same expression conditions. See FIG. 5C. Overexpression of EcHemH in this strain decreased CoPPIX production, suggesting that the dynamics of porphyrin metabolism may be affected by HemH overexpression. Satisfyingly, L13R HemH co-expression did yield improved CoPPIX relative to the parent EcHemH, although the effect was modest in this strain.

The surprisingly efficient incorporation of cobalt into PPIX by BL21(DE3) described here inspired testing the same method for the incorporation of other metals: copper, nickel, and zinc. Analogous to expression with cobalt, 1 mM of CuCl₂, NiCl₂, and ZnCl₂ were added to 1 L of culture expressing DyP in TB media at the time of induction. DyP was purified and the metalloporphyrin content interrogated by UPLC (data not shown). Zinc and copper supplemented cultures appeared to contain exclusively the native heme cofactor (FePPIX) and unmetallated PPIX (H2PPIX). The DyP resulting from expression with added nickel contained a mixture of FePPIX, H2PPIX, and the nickel-containing analog NiPPIX.

### Validation of a Method for Cobalt-Substituted Hemoproteins in Rich Media

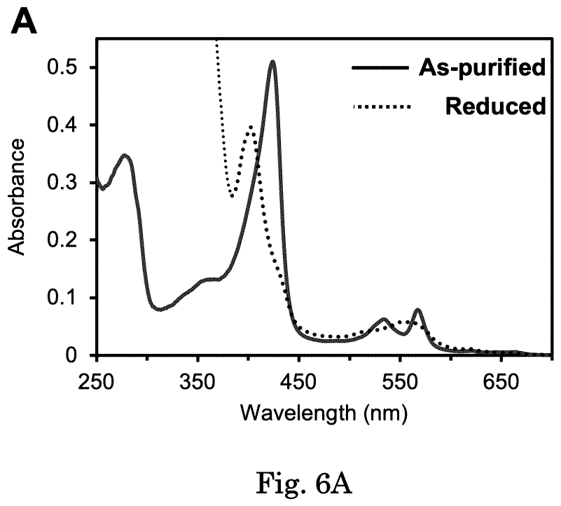

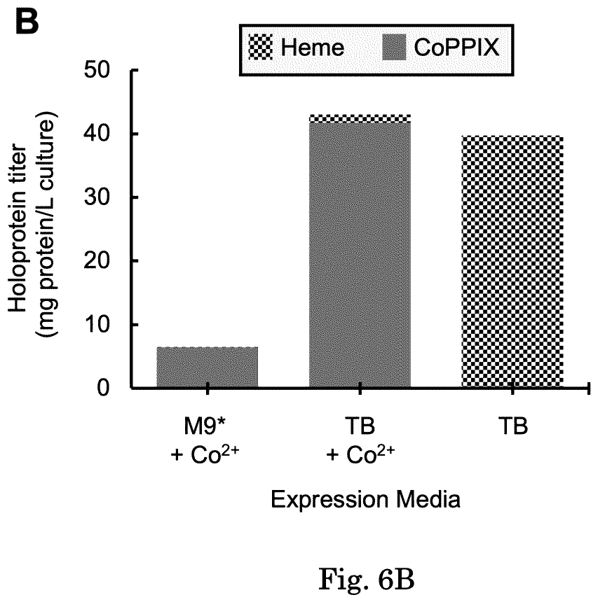

It was determined that the timing of adding 1 mM Co²⁺ to the media had no significant effect on the metalloporphyrin content and yield of holoprotein (data not shown). Consequently, a standard protocol was developed in which the metal is added at the time of induction, along with δALA, which was convenient and effective. With this method CoDyP was obtained at titer of 43 mg protein per L cell culture, and the resulting proteins were >95% cobalt loaded relative to iron. This yield represents a nearly 10-fold improvement in protein titer over the previous method in minimal media (Perkins, et al. “De Novo Biosynthesis of a Nonnatural Cobalt Porphyrin Cofactor in E. coli and Incorporation into Hemoproteins,” Proc. Natl. Acad. Sci. 2021, 118 (16), 2021), and gives similar titers to expression of the native heme-loaded protein. See FIGS. 6A and 6B.

The efficiency of cobalt-substituted hemoprotein production was then tested with a different protein scaffold, the P450 enzyme CYP119 from S. acidcaldarous under pET22b (IPTG-inducible) and pBAD (arabinose-inducible) expression systems. From 1 L of cell cultures in rich media 2 mg and 43 mg of ˜95% CoPPIX-loaded hemoprotein were obtained from the pET22b and pBAD systems, respectively. This expression compares favorably with that of the analogous heme proteins, which were obtained at 3 mg and 40 mg per L culture. Furthermore, this method requires virtually no additional steps or genetic manipulations relative to that of canonical heme protein, aside from the addition of an inexpensive cobalt salt (CoCl₂) at the time of induction.

Activity on Fe²⁺ and Co²⁺ was generally similar, indicating that EcHemH is an effective cobalt chelatase. To test the effect of chelatase metal specificity on the production of CoPPIX versus heme in BL21(DE3), an engineering campaign was undertaken to improve the Co²⁺ selectivity of the native BL21(DE3) ferrochelatase. The enzyme was surprisingly tolerant to mutation, as site saturation mutagenesis at seven of 10 active site residues yielded active chelatase variants. The L13R mutation causes a 30-fold shift in specificity toward cobalt insertion. The engineering of this variant enabled exploration how expression of this cobalt-favored chelatase affected the distribution of MPPIX products generated in vivo.

Notably—and surprisingly—it was found that the metal specificity of an over-expressed chelatase plays only a minor role in determining the distribution of metalated porphyrins in BL21(DE3). Large changes in Co²⁺ versus Fe²⁺ specificity, such as those exhibited by the variant L13R, do not outweigh the myriad other metabolic factors governing PPIX metalation. Indeed, it was found that the native promiscuity of EcHemH is already sufficient for efficient CoPPIX production in vivo.

Hemoproteins comprise a diverse family of widely studied and used biocatalysts. Often, BL21(DE3) E. coli is the organism used to produce these hemoproteins—and the cofactors they bear—at high titers. By studying the metalation and incorporation of a non-native heme analog, CoPPIX, the method provides insight into how biological metalation is controlled in this ubiquitous model organism.

The biosynthesis and incorporation of artificial cofactors is a long-standing and alluring challenge in the fields of synthetic biology and biocatalysis. New-to-nature cofactors may imbue enzymes with alternate modes of reactivity, but because these enzymes are difficult and tedious to produce, explorations of their potential activities are rare. The enantioselective carbene and nitrene transfer reactivity of metal-substituted hemoproteins, in particular, has led to the development of several strategies for incorporating pre-fabricated metalloporphyrins into hemoproteins. See, for example, Bloomer, et al. “Progress, Challenges, and Opportunities with Artificial Metalloenzymes in Biosynthesis,” Biochemistry 2023, 62 (2), 221-228. This method operates in rich media and thus enables further development of these unnatural, metallo-proteins.

### MATERIALS AND METHODS

All chemicals and chemical standards were purchased from commercial suppliers Sigma-Aldrich (St. Louis, Missouri), VWR (Radnor, Pennsylvania), Goldbio (St. Louis, Missouri), Frontier Biosciences (Hayward, California), and Fluka (Charlotte, North Carolina), and used without further purification. Casamino acids were obtained from New England Biosciences (Ipswich, Massachusetts) or Research Products International (Mt. Prospect, Illinois). Unless otherwise noted, all media and solutions were prepared using ultrapure water (≥18 MΩ, from a Thermo Scientific Barnstead Nanopure water purification system). Immobilized metal affinity chromatography resin or columns were purchased from GE Healthcare (Chicago, Illinois).

### Equipment and Instrumentation

New Brunswick I26R, 120 V/60 Hz shakers (Eppendorf North America, Enfield, Connecticut) were used for cell growth. When needed, the humidity was controlled with a HumidiKit™ Auto Humidity System for Incubators purchased from IncubatorWarehouse.com. Cell disruption was accomplished via sonication with a Sonic Dismembrator 550 (Fischer Scientific, Waltham, Massachusetts) sonicator. Electroporation (for transformation and cloning) was achieved using an Eppendorf E-porator set to 2500V. Optical density measurements of liquid cultures were recorded on a Ultrospec 10 cell density meter (Amersham Biosciences, Chicago, Illinois). Fast protein liquid chromatography was carried out using an ÄKTA Prime Plus (GE Healthcare). An Envision® 2105 multimode plate reader (Perkin Elmer, Shelton, Connecticut) was used to measure optical density in 96-well plates. UPLC/MS data were collected on an Acquity UPLC with an Acquity QDA MS detector (Waters Corporation, Framingham, Massachusetts). Electronic absorption data were collected on a UV-2600 Shimadzu (Long Beach, California) or a Varian (Palo Alto, California) Cary 4 Bio spectrophotometer, set to a spectral bandwidth of 0.5 nm. EPR spectra were collected with a Bruker (Billerica, Massachusetts) ELEXSYS E500 spectrometer equipped with an Oxford ESR 900 continuous flow liquid helium cryostat and an Oxford ITC4 temperature controller (Oxford Instruments, Abingdon, England). The microwave frequency was monitored using an EIP-brand model 625A continuous-wave microwave frequency counter (Phase Matrix, Inc., Santa Clara, California). ICPMS data were collected with a Shimadzu ICP-MS 2030 equipped with an AS-10 autosampler. Curve fitting for IC₅₀ assays was performed using Graphpad PRISM™ Software (Dotmatics, Boston, Massachusetts).

### Media Preparation and Cell Growth

Cells were grown in either Luria-Bertani (LB) broth, using the conventional recipe, or in supplemented M9 broth (M9*) using an adaptation of the recipe from Majtan et al. “Purification and characterization of cystathionine β-synthase bearing a cobalt protoporphyrin,” Arch. Biochem. Biophys. 508, 25-30 (2011). A stock solution of 10× M9 salts was prepared (75.2 g/L Na₂H₂PO₄, 30 g/L KH₂PO₄, NaCl 5 g/L, NH₄Cl 5 g/L), pH was adjusted to 8.0 with HCl or NaOH, and the solution was autoclaved. M9* media was assembled from 10× salts, 100× amino acid supplement stock (0.4 g/mL casamino acids, 1 mg/mL thiamine, sterile filtered), 100× glucose solution (20% glucose w/v, sterile filtered), 100 μM CaCl₂ (autoclaved as a 1 M aqueous solution), and 200 mM MgSO₄(autoclaved as a 1 M aqueous solution). The media was diluted to the appropriate volume and stored at room temperature until use. All cultures were inoculated from single colonies and incubated at 37° C. and 225 rpm unless otherwise stated. All growths of plasmid-containing bacteria were supplemented with 100 mg/L of ampicillin (Amp).

### General Cloning Procedures

Hemoprotein genes appended with C-terminal 6-His tags and flanked by regions complementary to a pET22b vector were purchased as codon optimized gBlocks from Integrated DNA Technologies. These DNA fragments were inserted into a pET22b vector by the Gibson Assembly method. (Gibson, D. G. et al. “Enzymatic assembly of DNA molecules up to several hundred kilobases,” Nat. Methods 6, 343-345 (2009).) The Gibson products were subsequently transformed into electrocompetent E. coli BL21(DE3) cells via electroporation. After 30-45 minutes of recovery in LB broth at 37° C. and 225 rpm, cells were plated onto LB plates with 100 mg/L Amp and incubated at 37° C. overnight. Single colonies were used to inoculate 5 mL of LB broth and grown overnight. Plasmid DNA was extracted and purified using a Zyppy™ Plasmid MiniPrep Kit (Zymogen Research, Irvine, California) and gene sequences were confirmed via Sanger sequencing (Functional Biosciences, Madison, Wisconsin). Purified plasmids were transformed into electrocompetent E. coli BL21(DE3) (New England Biosciences) using the above procedure.

### Assessing Effect of CoCl₂ Supplementation on E. coli Growth Profile

The growth of E. coli BL21(DE3) in M9* under various conditions was followed. Single colonies of vector-less BL21(DE3) were used to inoculate 5 mL starter cultures of antibiotic-free M9* broth in triplicate and were grown overnight at 37° C. and 225 rpm. These starter cultures (500 μL of each) were used to inoculate growth cultures (50 mL each in 250 mL baffled Erlenmeyer flasks), prepared from 50 ml of M9* media supplemented with 250 μM δ-aminolevulinic acid (δALA) (for δALA only, Fe+δALA, and Co+δALA growths) and 500 μM metal salt (FeCl₃ for Fe+δALA growth, CoCl₂ for Co+δALA growth). These cultures were grown at 37° C. and 225 rpm, measuring OD₆₀₀ every 1-3 hours. Growth was stopped and cells were harvested once the OD₆₀₀ did not significantly increase from one time point to the next. Of note, severely diminished growth was observed under all growth conditions when non-baffled flasks were used. Even after 3 days of incubation in non-baffled flasks (37°° C., 225 rpm), no growth was detected in the Co+δALA cultures (data not shown).

### Passaging E. coli Through CoCl₂ Supplemented Media

Three separate lineages of passaged E. coli were generated and nicknamed Alvin, Simon, and Theodore and which are denoted “A,” “S,” and “T,” respectively. A glycerol stock of BL21(DE3) E. coli (New England Biosciences) was streaked onto an antibiotic-free LB agar plate and three single colonies were used to inoculate 5 mL starter cultures of M9*. The cultures were shaken at 225 rpm at 37° C. for 16 h. Each starter culture (50 μL) was used to inoculate 5 mL of M9*, supplemented with 150 μM CoCl₂. To passage, these cultures were grown for 8-15 hr, after which 50 μL of each was used to inoculate 5 mL of fresh M9*, supplemented with 150 μM CoCl₂. This passaging was repeated every 8-15 h for a total of 15 passages over 2 weeks, with three changes in CoCl₂ concentration. After the 8^{th} passage, the concentration of CoCl₂ was increased to 450 μM. After the 12^{th} passage the concentration was increased to 750 μM, and after the 14^{th} passage the concentration was increased to 1 mM CoCl₂. OD₆₀₀ values (data not shown) were taken at the end of each growth period. The 15× passaged cultures (A15, S15, and T15 populations) were used to make 25% glycerol stocks and were stored at −80° C. until further use.

### Quantitation of E. coli Cobalt Tolerance with IC₅₀ Assays—Effect of Passaging on E. coli Cobalt Tolerance (IC₅₀)

The tolerance of parent and passaged E. coli BL21(DE3) cells to CoCl₂ was tested by measuring the IC₅₀ for CoCl₂ in various cell lines. Glycerol stocks of un-passaged BL21(DE3) E. coli (New England Biosciences) and passaged populations (A15, S15, and T15) were streaked onto antibiotic-free LB agar plates to obtain single clones. Three single colonies from each plate were used to inoculate 5 mL of antibiotic-free M9*, and the cultures were grown at 37° C. for 16 h. These cultures (5 uμof each) were used to inoculate 195 μL of M9* supplemented with variable amounts of CoCl₂ (0-3 mM) in triplicate in 96-well plates. Following inoculation, the 96-well plates were incubated at 37° C. with a shake speed of 180 rpm. To prevent excessive evaporation, plates were stacked with a water-filled plate above and below, and the incubator's humidity was maintained between 60-80% relative humidity with a humidifier. After 9 h, the optical density at 600 nm of each well was measured with a plate reader. To eliminate any contribution from the absorbance of or scattering from the CoCl₂ supplemented M9*, the OD₆₀₀ value for each growth well was corrected by subtracting the average optical density of 6 wells with the same CoCl₂ concentration in an identical plate that was inoculated with 5 μl sterile M9* and incubated with the rest of the plates. The OD₆₀₀ versus CoCl₂ concentration data were fit to the Hill equation (below):

$${OD}_{600} = Bottom + \frac{Top - Bottom}{1 + {( \frac{[ {CoCl}_{2} ]}{{IC}_{50}} )}^{Hill coefficient}}$$

Curve fits were generated (data not shown). Best fit curves for single clones of the passaged strains were superimposable, so the individual data points for passaged strains were aggregated and analyzed together. The best-fit IC₅₀ value for the 15× passaged strain was 10-fold higher than that of the parent BL21(DE3): 250 μM CoCl₂ (Hill coefficient=−50) versus 20 μM CoCl₂ for the un-passaged strain).

### Effects of Hemoprotein and Non-Heme-Protein Overexpression on E. coli Cobalt Tolerance (IC₅₀)

An experiment was run to determine whether expression of a heme-containing protein (Mb*) conferred greater tolerance to CoCl₂ than expression of a non-heme, cofactor-containing protein. Single-colony derived overnight cultures (5 μL of each) of BL21(DE3) E. coli bearing a pET22b plasmid encoding either Mb* or PfuTrpB (the PLP-dependent β subunit of tryptophan synthase from P. furiosus) were used to inoculate 195 μl of M9* with 100 mg/L Amp and variable amounts of CoCl₂ (0-3 mM) and IPTG (0-1000 mM) in a 96-well plate. Plates were incubated at 37° C. and 180 rpm for 9 h. Humidity was controlled to eliminate evaporation during incubation, as described above, after which OD₆₀₀ values were measured. The measured OD₆₀₀ values were corrected for absorbance of or scattering from CoCl₂-containing media as described above. Each starter culture inoculated three rows in the 96-well plate, yielding experimental triplicate measurement of OD₆₀₀. Data was processed as described above (data not shown).

### Comparing the Mb* Expression Efficiency Between E. coli BL21(DE3) and Passaged Strains in CoCl₂ Supplemented Media

The passaged, cobalt-tolerant strains were tested for their ability to produce CoMb*. Those results were then compared to the corresponding results for unpassaged BL21(DE3). Passaged population glycerol stocks (A15 and S15) and control BL21(DE3) cells were streaked onto antibiotic-free LB agar plates and grown at 37° C. Single colonies from each plate were grown in LB broth, and the cells were rendered electrocompetent by washing multiple times with 10% glycerol in deionized water. Purified pET22b plasmid bearing a C-terminal 6-His-tagged Mb* gene was transformed into these cells via electroporation according to the general cloning procedure given above. Starter cultures (a single colony into 5 mL M9*) were used to inoculate 50 ml of M9* broth in 250 mL Erlenmeyer flasks, which were incubated in a shaker at 37° C. and 225 rpm. When culture OD₆₀₀ values reached approximately 0.2, CoPPIX production was induced by addition of δALA and CoCl₂, to final concentrations of 200 μM and 500 μM, respectively. Incubation continued until the culture OD₆₀₀ values reached 0.6-0.8, at which time cultures were removed and placed on ice for 40 minutes. Mb* production was induced by addition of IPTG (to a final concentration of 1 mM), and the cultures were incubated at 23° C. and 140 rpm for 16 h. Cells were harvested by centrifugation (4,300×g, 1 h, 4° C.) and stored at −20° C. for 24 h before lysis and purification, as described below. Metalloporphyrin content of the purified Mb* stocks were assessed using the modified pyridine hemochrome assay described below.

### Hemoprotein Overexpression and Purification; Expression of Cobalt-Substituted Hemoproteins

CoPPIX-substituted hemoproteins were overexpressed in the following manner. M9* overnight starter cultures of E. coli bearing hemoprotein expression vectors described earlier were used to inoculate 1 L of M9* media containing 100 mg/L Amp. These cultures were incubated at 37° C. and 225 rpm. After the OD₆₀₀ reached 0.2-0.3, both CoCl₂ and δALA were added from sterile filtered 1.0 M stocks to final concentrations of 500 mM and 250 mM, respectively. The expression flasks were returned to the incubator at 37° C. and 180 rpm. Once the OD₆₀₀ had reached 0.6-0.7, the expression cultures were chilled on ice for 20-30 minutes. Expression was induced with 250 mM IPTG and the cultures were incubated overnight at 23° C. and 140 rpm. Cells were harvested by centrifugation at 4300×g and 4° C. Cells were stored at −20° C. until lysis (at least 12 hours and no more than 2 weeks).

### General Method for Protein Purification

Cell pellets derived from the expression procedure described above were lysed and proteins were purified according to the following procedure. Frozen cell pellets were resuspended in lysis buffer (50 mM potassium phosphate buffer, 250 mM NaCl, 10 mM imidazole, 1 mg/ml hen egg white lysozyme, 0.2 mg/ml DNase 1, 2 mM MgCl₂, pH 8.0). A volume of 4 mL of lysis buffer per gram of wet cell mass was used. After incubation for 30 minutes at 37° C. and 140 rpm, the cells were disrupted via sonication for 20 min on ice (0.8 second pulses followed by 0.2 second pause, at a power setting of 5). The resulting lysate was clarified of cellular debris via centrifugation at 75,000×g for 30 min. Supernatant was applied to a Ni-NTA gravity column or a 5 mL HisTrap column equilibrated with binding buffer (50 mM potassium phosphate, 250 mM sodium chloride, 10 mM imidazole, pH 8.0). The column was washed with approximately 3 column volumes 15:85, 25:75 and 30:70 (v/v) mixtures of elution buffer (50 mM potassium phosphate, 250 mM sodium chloride, 200 mM imidazole, pH 8.0) and binding buffer. Proteins were eluted with 100% elution buffer. Eluent was pooled and dialyzed into storage buffer (50 mM potassium phosphate, pH 8.0) overnight at 4° C. Dialyzed protein solutions were concentrated to <500 μL using Amicon® Ultra 15 mL Centrifugal Filters (Millipore-Sigma, Burlington, Massachusetts) at a molecular weight cutoff of 15 kDa for Mb* and 30 kDa for Oxd, DyP and CYP119. Final protein solutions were flash frozen in liquid nitrogen and stored at −80° C.

### Expression of FeDyP in Rich Media

DyP was expressed under iron-containing, rich media conditions for comparison of the heme (FePPIX) and H2PPIX loading to that of the CoPPIX-substituted variant. Overnight starter cultures (5 ml) of E. coli bearing a pET22b with the DyP gene in LB broth were used to inoculate 1 L of Terrific Broth (TB)+100 mg/L Amp. These cultures were incubated at 37° C. and 225 rpm. Once the OD₆₀₀ reached 0.2-0.3, cultures were supplemented with 250 mM δALA, and the expression flasks were returned to the incubator at 37° C. and 180 rpm. When the OD₆₀₀ had reached 0.6-0.7, the expression cultures were chilled on ice for 20-30 minutes. Expression was induced with 250 μM IPTG and the cultures were incubated overnight at 23° C. and 140 rpm. Cells were harvested by centrifugation at 4300×g at 4° C. Cell pellets were stored at −20° C. prior to protein purification, as described above.

### Spectroscopic Characterization of Purified CoPPIX-Substituted Hemoproteins Electronic Absorption Spectroscopy of CoPPIX-Substituted Hemoproteins

Electronic absorption spectra were taken to characterize the purified CoPPIX-substituted hemoproteins to compare their spectra with previous literature examples. Purified protein was mixed with argon-sparged 50 mM potassium phosphate buffer, pH 7.0 in septum-sealed cuvettes to ensure anaerobic conditions. Protein solutions were placed in the spectrometer at 35° C. using a Cary temperature control module for 5 minutes prior to collecting as-isolated spectra. Reduction was initiated via syringe addition of an anaerobic stock solution of sodium dithionite in buffer, to achieve a final concentration of 3-4 mM. Spectra were recorded every five minutes until absorbance changes were no longer observed.

### Electron Paramagnetic Resonance (“EPR”) Spectroscopy of Co(II) PPIX-Substituted Hemoproteins

EPR spectroscopy was used to characterize the coordination environment of cobalt in each Co(II) PPIX-substituted hemoprotein. Protein samples were prepared in Ar-sparged buffer (50 mM potassium phosphate, pH 7.0) in septum-sealed cuvettes. Sodium dithionite was added as an anaerobic stock solution in the same buffer via syringe, to a final concentration of 4 mM. Samples were incubated for 2 hours at 35° C.; complete reduction was confirmed via electronic absorption spectroscopy. Samples were brought into an anaerobic chamber and concentrated to 200 mL using 0.5 mL centrifugal filters (Amicon, Millipore-Sigma). Protein solutions were transferred to quartz EPR tubes via syringe, removed from the anaerobic chamber and immediately frozen in liquid nitrogen.

The CoPPIX concentration in the EPR samples varied from 100 to 270 mM. Spectra were recorded at 0.05024 mW microwave power, 40.96 s time constant, 40.96 s conversion time, 100 kHz modulation frequency, and 5 G modulation amplitude. These parameters were chosen to optimize the signal on the basis of a power saturation experiment (data not shown).

### Quantification of Porphyrin Content; UPLC-MS Analysis of Porphyrin Content for Cell Pellets and Purified Protein; Cell Pellet Sample Preparation

The porphyrin content of cell pellets grown under a variety of conditions was evaluated by UPLC-MS. Stationary phase cell cultures were obtained from the growth curve analysis. Cells were harvested by centrifugation (4,300×g, 5 min, 4° C.), and cell pellets were immediately lysed via addition of 2 mL of an 8:2 pyridine: 1.2 M HCl mixture and vortexed for 3 minutes. Saturated MgSO₄ (2 mL) was added, and lysates were centrifuged (4,300×g, 5 min, 4° C.) to yield defined aqueous and organic layers. The organic supernatant was removed and clarified by further centrifugation (14,000 rpm, 3 min). The clarified, organic-soluble portion of the cell lysate was then injected onto UPLC-MS. Reported porphyrin concentrations are the average of biological triplicate measurements, and the standard error for these measurements was propagated from the standard error of the calibration curve (data not shown).

### Purified Protein Sample Preparation

The relative porphyrin content of purified protein samples was analyzed by UPLC-MS. Purified protein samples, obtained from the protein purification procedure described above, were thawed on the benchtop. An aliquot of 2-30 μl purified protein was added to 200 μl 8:2 pyridine: 1.2 M HCl mixture and the resulting mixture was vortexed for 1 min. Saturated MgSO₄ (200 μL) was added and the solution was vortexed vigorously once more. The emulsion was centrifuged (4,300×g, 5 min, 4° C.) to yield defined aqueous and organic layers. The organic supernatant was removed and clarified by further centrifugation (14,000 rpm, 3 min). The clarified, organic-soluble portion of the cell lysate was injected onto UPLC-MS. Reported porphyrin concentrations are the average of triplicate extractions from a single protein sample, and the standard error for these measurements was propagated from the standard error of the calibration curve (data not shown).

### UPLC Analysis

The UPLC chromatographic method consisted of an isocratic mobile phase (25% water in acetonitrile with 0.1% formic acid), flowing at a rate of 1 mL/min and 40° C., over an ACQUITY UPLC-CSH Phenyl-Hexyl 1.7 μm column (Waters). A 3.0 μL injection volume was used. Clear porphyrin signals were observed via absorbance at the porphyrin Soret maximum (398 nm for FePPIX, 400 nm for H₂PPIX, and 423 for CoPPIX), and positive single ion reads (SIR) (616.3 m/z for FePPIX, 563.3 m/z for H₂PPIX, and 619.3 m/z for CoPPIX). Porphyrin content was quantified by comparison to standard curves. Integrated UV absorbance was used to quantify H₂PPIX and FePPIX. For CoPPIX, interfering absorption at 423 nm and the CoPPIX retention time rendered absorption analysis less useful for cell lysates. Therefore, CoPPIX concentration was assessed using integrated SIR for cell pellet analyses. The calculated concentration of porphyrin in the analyte solution and the volume of organic supernatant obtained by centrifugation were used to calculate the concentration of porphyrin in the original sample.

### ICP-MS Analysis for Total Metal Content of Purified Proteins

Metal content of each protein was determined by inductively coupled plasma-mass spectrometry. Metal samples were prepared by addition of trace analysis grade 70% nitric acid (350 mL) to protein stocks (<30 mL). Samples were incubated at 80° C. for 4 hours to ensure complete digestion and subsequently diluted with ultrapure water to give a final concentration of 5% nitric acid. Cobalt and iron standard solutions were prepared in 5% nitric acid solutions from 1000 ppm atomic absorption standards. Metal content was quantified by comparison to a standard curve (data not shown). To calculate CoPPIX-loading, cobalt concentrations were compared to protein concentrations measured using the Pierce™ bicinchoninic acid assay (BCA) assay kit (ThermoFisher Scientific) according to the manufacturer's instructions.

### Modified Pyridine Hemochrome Assay

A modified version of the pyridine hemochrome assay was used to qualitatively assess metalloporphyrin content in purified proteins. See Berry, et al. “Simultaneous determination of hemes a, b, and c from pyridine hemochrome spectra,” Anal. Biochem. 161, 1-15 (1987). Protein solution (50 mL) was added to 350 μl of pyridine hemochrome reagent (40 mL pyridine, 36 mL H₂O, and 4 mL 1 M NaOH) in a quartz cuvette. The sample was capped, mixed thoroughly, and heated to 70° C. to fully denature the protein and form the pyridine metalloporphyrin complex. A few crystals of sodium dithionite were added and the sample was cooled to 30° C. Spectra were recorded at 30° C. Comparison of these spectra to reduced pyridine adducts of commercially available Co(III)PPIX(CI) and Fe(III)PPIX(CI) yields qualitative assessment of metalloporphyrin content.

1. A method to make Co- or Ni-substituted protoporphyrin IX, the method comprising:
(a) incubating an E. coli strain in a rich medium containing iron ions and added Co²⁺ or Ni²⁺ ions, for a time and under conditions wherein the E. coli strain produces Co- or Ni-substituted protoporphyrin IX.

2. The method of claim 1, wherein in step (a) the E. coli strain is selected from the group consisting of E. coli BL21(DE3) and E. coli JM109(DE3).

3. The method of claim 1, wherein in step (a) the E. coli strain does not overexpress E. coli ferrochelatase (“EcHemH”).

4. The method of claim 1, wherein in step (a) the E. coli strain overexpresses EcHemH.

5. The method of claim 1, wherein step (a) comprises incubating the E. coli strain in a rich medium comprising from about 0.1 to about 3 mM Co²⁺ or Ni²⁺.

6. The method of claim 1, wherein step (a) comprises incubating the E. coli strain in a rich medium comprising δ-aminolevulinic acid.

7. The method of claim 1, wherein in step (a) the E. coli strain is transformed to contain and express an exogenous gene encoding EcHemH with a L13R mutation.

8. The method of claim 7, wherein step (a) comprises incubating an E. coli strain selected from the group consisting of E. coli BL21(DE3) and E. coli JM109(DE3).

9. The method of claim 1, further comprising (b) incorporating the Co- or Ni-substituted protoporphyrin IX into a polypeptide or protein.

10. A method to make Co- or Ni-substituted protoporphyrin IX, the method comprising:
(a) incubating an E. coli strain selected from the group consisting of E. coli BL21(DE3) and E. coli JM109(DE3) in a rich medium containing iron ions and added Co²⁺ or Ni²⁺ ions, for a time and under conditions wherein the E. coli strain produces Co- or Ni-substituted protoporphyrin IX.

11. The method of claim 10, wherein in step (a) the E. coli strain does not overexpress E. coli ferrochelatase (“EcHemH”).

12. The method of claim 10, wherein in step (a) the E. coli strain overexpresses EcHemH.

13. The method of claim 10, wherein step (a) comprises incubating the E. coli strain in a rich medium comprising from about 0.1 to about 3 mM Co²⁺ or Ni²⁺.

14. The method of claim 10, wherein step (a) comprises incubating the E. coli strain in a rich medium comprising δ-aminolevulinic acid.

15. The method of claim 10, wherein in step (a) the E. coli strain is transformed to contain and express an exogenous gene encoding EcHemH with a L13R mutation.

16. A method to make Co- or Ni-substituted protoporphyrin IX, the method comprising:
(a) incubating an E. coli strain selected from the group consisting of E. coli BL21(DE3) and E. coli JM109(DE3) in a rich medium containing iron ions and added Co²⁺ or Ni²⁺ ions, wherein the E. coli strain has been transformed to contain and express an exogenous gene encoding EcHemH with a L13R mutation, for a time and under conditions wherein the E. coli strain produces Co- or Ni-substituted protoporphyrin IX.

17. The method of claim 16, wherein step (a) comprises incubating the E. coli strain in a rich medium comprising δ-aminolevulinic acid.

18. An E. coli host transformed to contain and express a gene construct encoding a mutant EcHemH including a L13R mutation.

19. The E. coli host of claim 18, wherein the E. coli is selected from the group consisting of E. coli BL21(DE3) and E. coli JM109(DE3).
